(19) United States
(12) Patent Application Publication
(10) Pub. No.: US 2026/0207641 A1
(43) Pub. Date: Jul. 23, 2026

(54) COMBINATION THERAPY FOR CANCER USING ROS-ACTIVATED PRODRUGS AND ROS-AMPLIFYING THERAPEUTICS

(71) Applicant: UWM Research Foundation, Inc., Milwaukee, WI (US)

(72) Inventors: Xiaohua Peng, Milwaukee, WI (US); Taufeeque Ali, Milwaukee, WI (US)

(21) Appl. No.: 19/140,565

(22) PCT Filed: Jan. 17, 2024
(86) PCT No.: PCT/US2024/011722

§ 371 Date: Jun. 18, 2025

Related U.S. Application Data
(60) Provisional application No. 63/480,347, filed on Jan. 18, 2023.

(51) Int. Cl. A61K 31/69; A61K 31/235; A61K 31/355; A61K 31/375; A61K 31/4412; A61K 31/704; A61K 33/243; A61K 33/36; A61K 38/14; A61K 45/06; A61P 35/00

(52) U.S. Cl. CPC A61K 31/69; A61K 31/235; A61K 31/355; A61K 31/375; A61K 31/4412; A61K 31/704; A61K 33/243; A61K 33/36; A61K 38/14; A61K 45/06; A61P 35/00

(57) ABSTRACT

Described herein is an anticancer and anti-inflammatory combination therapy comprising one or more antineoplastic agents and one or more pro-oxidants. In one embodiment, the antineoplastic agent is not active in the absence of the pro-oxidant. In another embodiment, the pro-oxidant is administered prior to the antineoplastic agent to induce targeted activity of the antineoplastic agent.

### CROSS-REFERENCE TO RELATED APPLICATIONS

This application claims priority to U.S. Provisional Patent Application No. 63/480,347 filed on Jan. 18, 2023, which is incorporated by reference herein in its entirety.

### FEDERALLY SPONSORED RESEARCH

This invention was made with government support under grant number CA277656 awarded by the National Cancer Institute. The government has certain rights in the invention.

### BACKGROUND

The urgency for efficacious anticancer agents is underscored by cancer's status as the second most common global and US cause of death. However, the intricate nature of cancers, marked by intricate cellular pathways, diverse drug resistance mechanisms, and metabolic variations, present significant obstacles for achieving desired and enduring treatment outcomes through single-agent therapies. Single agents often yield only transient and incomplete responses due to cancer's constant evolution, resulting in target changes, drug resistance, and progression. In animal models, combining multiple anticancer agents has proven more effective than single-agent approaches. Despite promising data, around 90% of anticancer agents assessed as standalone treatments in clinical trials fail FDA approval. Therefore, prioritizing combination therapies' exploration during early testing is vital, as they hold future FDA approval potential.

Designing effective combination therapies poses numerous research challenges. Selecting a complementary anticancer agent that enhances efficacy without increasing off-target effects is complex. Identifying agents suitable for combined evaluation and measurement is another hurdle. Balancing mechanisms of action, safety profiles, and dosing regimens when combining agents within a class further complicates matters. Moreover, collaborations among labs developing multiple anticancer agents can bring economic, intellectual property, and logistical challenges. The funding of research projects by the government and private sector underscore that the benefits and safety of combination anticancer agents' outweigh production costs. Identifying combination agents offering significant benefits while minimizing complexities is crucial.

Ascorbic acid has well-established properties known for combating cancer. It triggers cytotoxicity by producing substantial amounts of hydrogen peroxide (H₂O₂), leading to the selective killing of cancer cells. The vulnerability of cancer cells to H₂O₂ is linked to their lower catalase (CAT) levels, a trait commonly found among cancer cells but not in normal ones. A wealth of scientific literature has documented the selective targeting of cancer cells through H₂O₂-induced cytotoxicity. The intricate role of generating reactive oxygen species (ROS) in shaping cancer's growth, progression, and metastasis underscores the complexity of this phenomenon, warranting meticulous examination. Ascorbic acid's behavior as both a prooxidant and antioxidant is context dependent. The impact of hydrogen peroxide (H₂O₂) on cancer cells varies due to their concentrations and conditions. Researchers have identified the potential advantages of adjusting ROS levels to heighten oxidative stress within cancer cells, ultimately prompting cell death.

Reports indicate that pharmacological ascorbic acid concentrations (mM scale) induce extracellular H₂O₂ accumulation in cancer cells, which penetrates cells, generating hydroxyl radicals and causing DNA damage, ultimately leading to cell death. However, ascorbic acid's prooxidant role has been challenged by clinical studies administering high oral doses to advanced cancer patients. IV and IP ascorbic acid administration achieves concentrations as high as 20 mM, unattainable orally. Oral ascorbic acid remains within physiological concentrations.

The utilization of ascorbic acid by medical professionals and those in complementary medicine is met with skepticism and lacks substantial clinical evidence of its effectiveness. Numerous ongoing clinical trials investigate ascorbic acid's efficacy in cancer treatment. It has been suggested that high-dose ascorbic acid can be a beneficial addition to chemotherapy, potentially mitigating chemotherapy-related adverse effects in some cases. However, despite its widespread usage and potential drawbacks, the utilization of ascorbic acid as an anticancer treatment is not comprehensively understood. Its underlying mechanisms have been explored in various research studies. Due to the limited understanding of its mechanism of action, further research is needed to fully comprehend its potential applications.

What are needed are compositions and methods for treating cancer comprising reactive oxygen species-activated prodrugs and reactive oxygen species-amplifying therapeutics.

### SUMMARY

One embodiment described herein is a combination therapeutic comprising: a therapeutically effective amount of one or more antineoplastic agents, or pharmaceutically acceptable salts or esters thereof, and a therapeutically effective amount of one or more pro-oxidants, or pharmaceutically acceptable salts or esters thereof. In one aspect, the one or more antineoplastic agents is an alkylating agent or a pro-drug of an alkylating agent. In another aspect, the one or more antineoplastic agents has a structure:

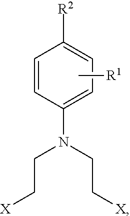

wherein
X, at each occurrence, is independently halo;
R¹, at each occurrence, is independently hydrogen, C₁₋₆alkyl, C₁₋₆alkylene, C₁₋₆haloalkyl, cyano, —OR^{1a}, —SR^{1a}, —CO₂R^{1a}, —C(O)R^{1a}, —SO₂R^{1b}, —N(R^{1b})₂, —CO₂N(R^{1b})₂, or —NO₂;
R² is BH(OR^{1b}), B(OR^{1b})₂,

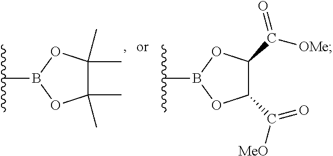

R^{1a}, at each occurrence, is independently hydrogen, C₁₋₆alkyl, or C₁₋₂haloalkyl; and
R^{1b}, at each occurrence, is independently hydrogen or C₁₋₆alkyl.

In another aspect, the one or more antineoplastic agents is FAN-NM-CH₃. In another aspect, the one or more antineoplastic agents is a DNA crosslinking agent or a pro-drug of a DNA crosslinking agent. In another aspect, the one or more antineoplastic agent is selected from the group comprising:

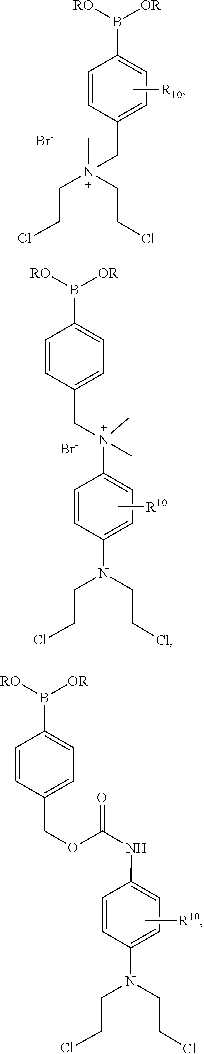

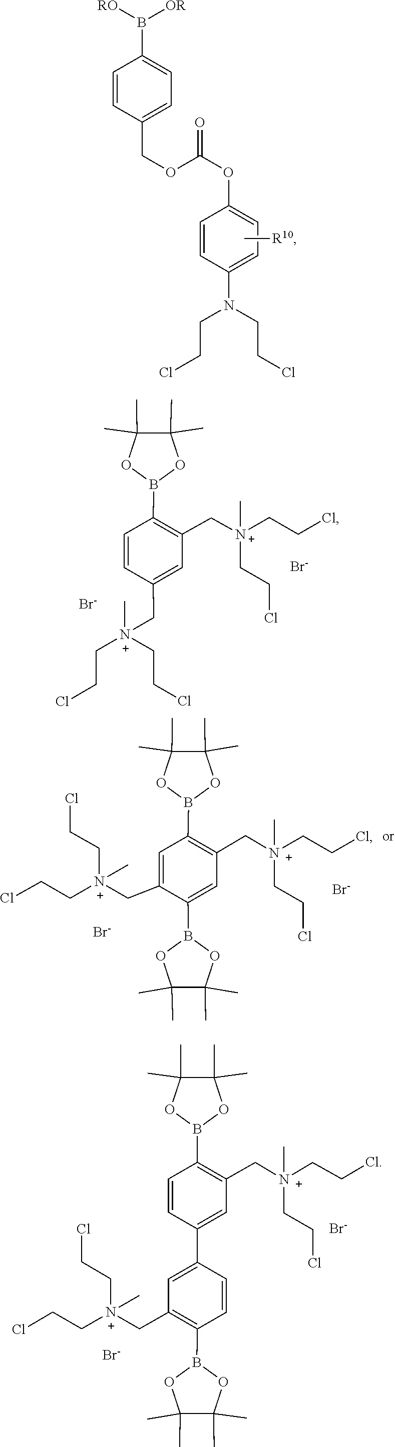

wherein R¹⁰, at each occurrence, is independently hydrogen, C₁₋₆alkyl, C₁₋₆alkylene, C₁₋₆haloalkyl, cyano, —OR^{1a}, —SR^{1a}, —CO₂R^{1a}, —C(O)R^{1a}, —SO₂R^{1b}, —N(R^{1b})₂, —CO₂N(R^{1b})₂, —NO₂, or —N(RR^{1b})—OR^{1a};
R^{1a}, at each occurrence, is independently hydrogen, C₁₋₆alkyl, or C₁₋₂haloalkyl; and
R^{1b}, at each occurrence, is independently hydrogen or C₁₋₆alkyl.

In another aspect, the one or more antineoplastic agents comprise one or more chemotherapeutic agents. In another aspect, the one or more chemotherapeutic agents comprise adriamycin, anthracyclines, bleomycin, or cisplatin, their H₂O₂-activated prodrugs, or combinations thereof. In another aspect, the one or more pro-oxidants is a reactive oxygen species amplifying agent. In another aspect, the one or more pro-oxidants comprise compounds containing quinone moieties.

In another aspect, the one or more pro-oxidants comprise vitamin C, polyphenol, hydrogen peroxide, carotenoids (Lutein, β-carotene, Astaxanthin, Fucoxanthin, β-Cryptoxanthin, Bixin, and lycopene), diallyl trisulfide (DATS), indomethacin (indo), Piperlongumine, Vitamine E, β-Lapachone, Plumbagin, Arsenic Trioxide, Quercetin, Cinnamaldehyde, Bisdemethoxycurcumin (Curcumin), (−)-Epigallocatechin gallate, Gensenosides (Rg3, Rh2), 2-methoxyestradiol, Emodin, β-phenylethyl isothiocyanate, nonsteroidal anti-inflammatory drugs (NSAIDs), or combinations thereof. In another aspect, the therapeutically effective amount of the one or more antineoplastic agents is 0.001-200 mg/kg. In another aspect, the therapeutically effective amount of the one or more pro-oxidants is 1-20000 mg/kg.

Another embodiment described herein is a method for treating a disease or disorder, the method comprising: sequentially or simultaneously administering to a subject in need thereof a therapeutically effective amount of one or more antineoplastic agents, or pharmaceutically acceptable salts or esters thereof, and a therapeutically effective amount of one or more pro-oxidants, or pharmaceutically acceptable salts or esters thereof; and repeating the administration until the disease or disorder is treated, ameliorated, or symptoms are reduced. In one aspect, the disease or disorder is a cancer. In another aspect, the disease or disorder is a solid cancer. In another aspect, the cancer is a breast cancer, a glioblastoma, leukemia, lung cancer, or renal cancer. In another aspect, the disease or disorder is a cancer associated with oxidative stress. In another aspect, the disease or disorder is an inflammatory disease. In another aspect, the inflammatory disease is arthritis. In another aspect, administering comprises intraperitoneal injection, intramuscular injection, subcutaneous injection, intravenous injection, intrathecal infusion, oral administration, or a combination thereof. In another aspect, the one or more pro-oxidants increase an amount of reactive oxygen species in a cancerous cell. In another aspect, the one or more antineoplastic agents is active in the presence of reactive oxygen species. In another aspect, a therapeutically effective amount of a one or more pro-oxidants and one or more antineoplastic agents reduces a malignant neoplasm size, volume, mass, or a combination thereof. In another aspect, the one or more pro-oxidants is added at a period of time prior to addition of the antineoplastic agent. In another aspect, the period of time is 1-3 hours.

Another embodiment described herein is the use of one or more antineoplastic agents and one or more pro-oxidants, or pharmaceutically acceptable salts or esters thereof, as a medicament for the treatment of cancer or an inflammatory disease in subject in need thereof.

Another embodiment described herein is a kit comprising: one or more antineoplastic agents, or pharmaceutically acceptable salts or esters thereof; one or more pro-oxidants, or pharmaceutically acceptable salts or esters thereof; optionally a device or means for administering the antineoplastic agent and pro-oxidant; optionally tamper resistant packaging; and optionally, a label or instructions for use thereof.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A-B show dose-response curves of pro-oxidants. FIG. 1A shows the dose-response curves of pro-oxidants in MCF 10A (normal cells). FIG. 2A shows the dose-response curves of pro-oxidants in MDA-MB 468 (cancer cells).

FIG. 2 shows an IC₅₀ comparison between different pro-oxidants and FAN-NM-CH₃ in MCF 10A (normal cells) and MDA-MB-468 (cancer cells).

FIG. 3A-B show cytotoxicity in various cells lines. FIG. 3A shows the dose-response cytotoxicity of Vitamin C when incubated for 48 h in various cancer cell lines (n=3, the IC₅₀ values were determined by a nonlinear regression). FIG. 3B shows the dose-dependent cytotoxicity of the FAN-NM-CH₃, both alone and in combination with vitamin C in different cancer cells and normal cells when incubated for 48 h (n=3, the IC₅₀ values were determined by a nonlinear regression).

FIG. 4A-B show graphical representations of ascorbic acid treatment that selectively generates H₂O₂ and activates FAN-NM-CH₃ in cancer cells. FIG. 4A shows untreated cells. FIG. 4B shows treated cells.

FIG. 5A-B show catalase activity across different cancerous and non-cancerous cells. FIG. 5A shows catalase activity for different cell lines were determined using Catalase Colorimetric Activity Kit from Invitrogen (EIACATC). 1×10⁶ cells were used to prepare 1 mL cell lysate to evaluate the activity of catalase in MDA-MB-468, MCF7, U-87 MG, and MCF 10A cells. All samples were prepared in triplicate experiment and shown as mean±SD. FIG. 5B shows H₂O₂ levels in MDA-MB-468, MCF7, U-87 MG, and MCF 10A cells.

FIG. 6A-C show extracellular H₂O₂ levels in cancerous and non-cancerous cells measured by Amplex Red Assay (data are represented as mean±SD from three independent experiments) (Note: H₂O₂ control concentrations were adjusted by 2 folds to account for the 1:1 dilution of equal volume of the assay). FIG. 6A shows ascorbic acid concentration dependent H₂O₂ release in MDA-MB-468, MCF7, U-87 MG, and MCF 10A cells when incubated for 48 hours. FIG. 6B shows a H₂O₂ standard curve. FIG. 6C shows H₂O₂ release in samples treated with varied conditions over 48 hours.

FIG. 7A-D show intracellular H₂O₂ levels in cancerous and non-cancerous cells measured by AbGreen indicator (Abcam, ab138874) (data are represented as mean±SD from three independent experiments). Note: H₂O₂ standard curve concentrations were adjusted by 2 folds to account for the 1:1 dilution of equal volume of the assay. FIG. 7A shows ascorbic acid concentration dependent intracellular H₂O₂ levels in MDA-MB-468, MCF7, U-87 MG, and MCF 10A cells when incubated for 48 hours. FIG. 7B shows a H₂O₂ standard curve. FIG. 7C shows intracellular H₂O₂ levels in samples treated with varied conditions over 48 hours. FIG. 7D shows fluorescent cell staining of cells.

FIG. 8 shows DNA damage detected by alkaline comet assays in MCF 10A, MDA-MB-468, MCF7, and U-87 MG cells.

FIG. 9A-E show Comet assay analyses demonstrating a synergistic effect between ascorbic acid and FAN-NM-CH₃ in MDA-MB-468, MCF7, U-87 MG, and MCF 10A cells when incubated for 48 hours. The data are presented as the mean±SD (n=3). FIG. 9A shows head DNA (%). FIG. 9B shows Tail DNA (%). FIG. 9C shows Tail Moment. FIG. 9D shows Tail Olive Moment. FIG. 9E shows comet Images analyzed by TriTek CometScore Software.

FIG. 10A-B show the negative controls for the dosing safety study as it relates to body weight. FIG. 10A shows the amount (grams) of mice body weight observed over 5-day treatment and 2-days post treatment for delayed onset toxicity with vehicle (n=3). FIG. 10B shows the percent change (%) in mice weight observed over 5-day treatment and 2-days post treatment for delayed onset toxicity with vehicle (n=3).

FIG. 11A-B show the change in body weight in response to 5 mg/kg of FAN-NM-CH₃ (Pdrug) and Vitamin C (V C). FIG. 11A shows the amount (grams) of mice body weight observed during a 9-day period of dose-escalating Vitamin C in combination with 5 mg/kg of FAN-NM-CH₃ (prodrug) (n=3). FIG. 11B the percent change (%) in mice weight observed during a 9-day period of dose-escalating Vitamin C in combination with 5 mg/kg of FAN-NM-CH₃ (prodrug) (n=3).

FIG. 12A-B show the change in body weight in response to a standard dose of FAN-NM-CH₃ (Pdrug) and various doses of Vitamin C (V C). FIG. 12A shows the amount (grams) of mice body weight observed over 5-day treatment and 2-days post treatment for delayed onset toxicity with 500 mg/kg, 750 mg/kg, and 1 g/kg doses of Vitamin C in combination with 5 mg/kg of prodrug (n=3). FIG. 12B shows the percent change (%) in mice weight observed over 5-day treatment and 2-days post treatment for delayed onset toxicity with 500 mg/kg, 750 mg/kg, and 1 g/kg doses of Vitamin C in combination with 5 mg/kg of FAN-NM-CH₃ (prodrug) (n=3).

FIG. 13A-B show the change in body weight in response to 10 mg/kg of FAN-NM-CH₃ (Pdrug) and Vitamin C (V C). FIG. 13A shows the amount (grams) of mice body weight observed during a 9-day period of dose-escalating Vitamin C in combination with 10 mg/kg of FAN-NM-CH₃ (n=3). FIG. 13B shows the percent change (%) in mice weight observed during a 9-day period of dose-escalating Vitamin C in combination with 10 mg/kg of FAN-NM-CH₃ (n=3).

FIG. 14A-B show the change in body weight in response to 10 mg/kg of FAN-NM-CH₃ (Pdrug) and various doses of Vitamin C (V C). FIG. 14A shows the amount (grams) of mice body weight observed over 5-day treatment and 2-days post treatment for delayed onset toxicity with 500 mg/kg, 750 mg/kg, and 1 g/kg doses of Vitamin C in combination with 10 mg/kg of FAN-NM-CH₃ (n=3). FIG. 14B shows the percent change (%) in mice weight observed over 5-day treatment and 2-days post treatment for delayed onset toxicity with 500 mg/kg, 750 mg/kg, and 1 g/kg doses of Vitamin C in combination with 10 mg/kg of FAN-NM-CH₃ (n=3).

FIG. 15A-B show the change in body weight in response to 20 mg/kg of FAN-NM-CH₃ (Pdrug) and Vitamin C (V C). Treatment ended on day 3 due to significant weight loss and mice death with 3 g/kg Vitamin C dose in combination. FIG. 15A shows the amount (grams) of mice body weight observed over 5-day treatment with dose-escalating Vitamin C in combination with 20 mg/kg of FAN-NM-CH₃ (n=3). FIG. 15B shows the percent change (%) in mice weight observed over 5-day treatment with dose-escalating Vitamin C in combination with 20 mg/kg of FAN-NM-CH₃ (n=3).

FIG. 16A-B show the change in body weight in response to 20 mg/kg of FAN-NM-CH₃ (Pdrug) and various doses of Vitamin C (V C). FIG. 16A shows the amount (grams) mice body weight observed over 5-day treatment and 2-days post treatment for delayed onset toxicity with 500 mg/kg, 750 mg/kg, and 1 g/kg doses of Vitamin C in combination with 20 mg/kg of prodrug (n=3). FIG. 16B shows the percent change (%) in mice weight observed over 5-day treatment and 2-days post treatment for delayed onset toxicity with 500 mg/kg, 750 mg/kg, and 1 g/kg doses of Vitamin C in combination with 20 mg/kg of prodrug (n=3).

FIG. 17A-B show the time-dependent change in body weight for xenograft mice receiving intraperitoneal (IP) administrations of vehicle, 500 mg/kg Vitamin C, 3 mg/kg FAN-NM-CH₃ and the sequential doses of 500 mg/kg Vitamin C followed by 3 mg/kg FAN-NM-CH₃ 1 hour after the Vitamin C dose. FIG. 17A shows the amount (grams) of mice weight observed following treatment. FIG. 17B shows the percent change (%) in mice weight following treatment.

FIG. 18A-B show the time dependent tumor growth measure by caliper in the xenograft model of mice receiving intraperitoneal (IP) administration of vehicle, 500 mg/kg Vitamin C, 3 mg/kg kg FAN-NM-CH₃ and the sequential doses of 500 mg/kg Vitamin C followed by 3 mg/kg FAN-NM-CH₃ 1 hour after the Vitamin C dose. FIG. 18A shows the volume (mm³) of tumor growth following treatment. FIG. 18B shows the percent change (%) of tumor volume following treatment.

### DETAILED DESCRIPTION

Unless otherwise defined, all technical and scientific terms used herein have the same meaning as commonly understood by one of ordinary skill in the art. For example, any nomenclatures used in connection with, and techniques of biochemistry, molecular biology, immunology, microbiology, genetics, cell and tissue culture, and protein and nucleic acid chemistry described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms “amino acid,” “nucleotide,” “polynucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein.

As used herein, terms such as “include,” “including,” “contain,” “containing,” “having,” and the like mean “comprising.” The present disclosure also contemplates other embodiments “comprising,” “consisting essentially of,” and “consisting of” the embodiments or elements presented herein, whether explicitly set forth or not. As used herein, “comprising,” is an “open-ended” term that does not exclude additional, unrecited elements or method steps. As used herein, “consisting essentially of” limits the scope of a claim to the specified materials or steps and those that do not materially affect the basic and novel characteristics of the claimed invention. As used herein, “consisting of” excludes any element, step, or ingredient not specified in the claim.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “and/or” refers to both the conjunctive and disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to +10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “~” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to +10% of any value within the range or within 3 or more standard deviations, including the end points, or as described above in the definition of “about.”

As used herein, the terms “active ingredient” or “active pharmaceutical ingredient” refer to a pharmaceutical agent, active ingredient, compound, or substance, compositions, or mixtures thereof, that provide a pharmacological, often beneficial, effect.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

As used herein, the term “dose” denotes any form of an active ingredient formulation or composition, including cells, that contains an amount sufficient to initiate or produce a therapeutic effect with at least one or more administrations. “Formulation” and “composition” are used interchangeably herein.

As used herein, the term “prophylaxis” refers to preventing or reducing the progression of a disorder, either to a statistically significant degree or to a degree detectable by a person of ordinary skill in the art.

As used herein, the terms “effective amount” or “therapeutically effective amount,” refers to a substantially non-toxic, but sufficient amounts of an action, agent, composition, or cell(s) being administered to a subject that will prevent, treat, or ameliorate to some extent one or more of the symptoms of the disease or condition being experienced or that the subject is susceptible to contracting. This term as used herein may also refer to a dosage of a compound, compounds, or compositions that elicit a desire effect. The desired effect can be the reduction or alleviation of the signs, symptoms, or causes of a disease, or any other desired alteration of a biological system. An effective amount may be based on factors individual to each subject, including, but not limited to, the subject's age, size, type or extent of disease, stage of the disease, route of administration, the type or extent of supplemental therapy used, ongoing disease process, and type of treatment desired. This term as used herein may also refer to an amount effective at bringing about a desired in vivo effect in an animal, mammal, or human, such as reducing proliferation of a cancer cell.

As used herein, the term “subject” refers to an animal. Typically, the subject is a mammal. A subject also refers to primates (e.g., humans, male or female; infant, adolescent, or adult), non-human primates, rats, mice, rabbits, pigs, cows, sheep, goats, horses, dogs, cats, fish, birds, and the like. In one embodiment, the subject is a primate. In one embodiment, the subject is a human.

As used herein, a subject is “in need of treatment” if such subject would benefit biologically, medically, or in quality of life from such treatment. A subject in need of treatment does not necessarily present symptoms, particular in the case of preventative or prophylaxis treatments.

As used herein, the terms “inhibit,” “inhibition,” or “inhibiting” refer to the reduction or suppression of a given biological process, condition, symptom, disorder, or disease, or a significant decrease in the baseline activity of a biological activity or process.

As used herein, “treatment” or “treating” refers to prophylaxis of, preventing, suppressing, repressing, reversing, alleviating, ameliorating, or inhibiting the progress of biological process including a disorder or disease, or completely eliminating a disease. A treatment may be either performed in an acute or chronic way. The term “treatment” also refers to reducing the severity of a disease or symptoms associated with such disease prior to affliction with the disease. “Repressing” or “ameliorating” a disease, disorder, or the symptoms thereof involves administering a cell, composition, or compound described herein to a subject after clinical appearance of such disease, disorder, or its symptoms. “Prophylaxis of” or “preventing” a disease, disorder, or the symptoms thereof involves administering a cell, composition, or compound described herein to a subject prior to onset of the disease, disorder, or the symptoms thereof. “Suppressing” a disease or disorder involves administering a cell, composition, or compound described herein to a subject after induction of the disease or disorder thereof but before its clinical appearance or symptoms thereof have manifest.

“Administration” or “administering,” as used herein, refers to providing, contacting, and/or delivery of a compound or compounds by any appropriate route to achieve the desired effect. Administration may include, but is not limited to, oral, sublingual, parenteral (e.g., intravenous, subcutaneous, intracutaneous, intramuscular, intraarticular, intraarterial, intrasynovial, intrasternal, intrathecal, intralesional or intracranial injection), transdermal, topical, buccal, rectal, vaginal, nasal, ophthalmic, via inhalation, and implants.

As used herein “ROS” refers to reactive oxygen species.

As used herein “IC₅₀” refers to the concentration of inhibitor required to produce 50% inhibition.

“Pro-oxidant,” as used herein, refers any compound capable of increasing oxidative stress.

“Reactive oxygen species amplifying agent,” as used herein, specifically refers to any compound capable of increasing a reactive oxygen species. Non-limiting examples of reactive oxygen species include superoxide anion, hydrogen peroxide, and hydroxy radical.

“Antineoplastic agent,” as used herein, refers to compounds capable of ceasing cell growth. An antineoplastic agent is any compound with therapeutic utility in the treatment of diseases characterized by abnormal cell growth. Non-limiting examples of disease include neoplasms and cancer. This term as used herein may also refer to chemotherapeutic agents. An antineoplastic agent may exist as a pharmaceutically acceptable salt, prodrug, or prodrug of a prodrug. Non-limiting examples of antineoplastic agents and chemotherapeutics include alkylating agents, such as nitrogen mustards (for example, chlorambucil, chlormethine, cyclophosphamide, ifosfamide, and melphalan), nitrosoureas (for example, carmustine, fotemustine, lomustine, and streptozocin), platinum compounds (for example, carboplatin, cisplatin, oxaliplatin, and BBR3464), busulfan, dacarbazine, mechlorethamine, procarbazine, temozolomide, thiotepa, and uramustine; antimetabolites, such as folic acid (for example, methotrexate, pemetrexed, and raltitrexed), purines (for example, cladribine, clofarabine, fludarabine, mercaptopurine, and thioguanine), pyrimidines (for example, capecitabine), cytarabine, fluorouracil (e.g., 5-FU), and gemcitabine; plant alkaloids, such as podophyllum (for example, etoposide, and teniposide), taxane (for example, docetaxel and paclitaxel), vinca (for example, vinblastine, vincristine, vindesine, and vinorelbine); cytotoxic/antitumor antibiotics, such as anthracycline family members (for example, daunorubicin, doxorubicin, epirubicin, idarubicin, mitoxantrone, pirarubicin, vosaroxin, valrubicin, and mitoxantrone), bleomycin, hydroxyurea, geldan amycin, 17-N-allylamino-17-demethoxygeldanamycin (17 AAG), 17-dimethylaminoethylamino-17 demethoxygeldanamycin (17-DMAG), and mitomycin; topoisomerase inhibitors, such as camptothecin, 10-hydroxycamptothecin, irinotecan, SN-38, topotecan, rebeccamycin, Adriamycin, and etoposide.

“Prodrug” refers to a derivative of an active agent that requires a transformation within the body or cell to release the active agent. In certain embodiments, the transformation is an enzymatic transformation. In certain embodiments, the transformation is activated by a tumor-specific environment. Prodrugs are frequently, although not necessarily, pharmacologically inactive until converted to the active agent.

As used herein “FAN-NM-CH₃” refers to (4-(bis(2-chloroethyl)amino)-2-methylphenyl) boronic acid, the prodrug having the structure:

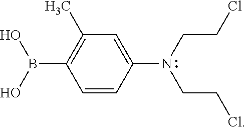 [SMILES: Cc1cc(N(CCCl)CCCl)ccc1B(O)O]

Upon activation, FAN-NM-CH₃ is converted to the active form, 4-(bis(2-chloroethyl)amino)-2-methylphenol,

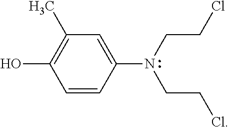

As used herein, an antineoplastic agent has the structure:

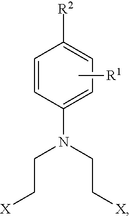

wherein
X, at each occurrence, is independently halo;
R¹, at each occurrence, is independently hydrogen, C₁₋₆alkyl, C₁₋₆alkylene, C₁₋₆-haloalkyl, cyano, —OR^{1a}, —SR^{1a}, —CO₂R^{1a}, —C(O)R^{1a}, —SO₂R^{1b}, —N(R^{1b})₂, —CO₂N(R^{1b})₂, or —NO₂;
R² is BH(OR^{1b}), B(OR^{1b})₂,

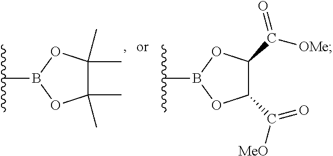

R^{1a}, at each occurrence, is independently hydrogen, C₁₋₆alkyl, or C₁₋₂haloalkyl; and
R^{1b}, at each occurrence, is independently hydrogen or C₁₋₆alkyl.

The structures and the syntheses of the antineoplastic agents described above are disclosed in U.S. Pat. No. 8,962,670, which is incorporated by reference herein for the teachings thereof.

Definitions of specific functional groups and chemical terms are described in more detail below. For purposes of this disclosure, the chemical elements are identified in accordance with the Periodic Table of the Elements, CAS version, Handbook of Chemistry and Physics, 75th Ed., inside cover, and specific functional groups are generally defined as described therein.

Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5th Edition, John Wiley & Sons, Inc., New York, 2001; Larock, Comprehensive Organic Transformations, VCH Publishers, Inc., New York, 1989; Carruthers, Some Modern Methods of Organic Synthesis, 3rd Edition, Cambridge University Press, Cambridge, 1987; the entire contents of each of which are incorporated herein by reference.

The term “acyl” refers to an alkylcarbonyl, cycloalkylcarbonyl, heterocyclylcarbonyl, arylcarbonyl or heteroarylcarbonyl substituent, any of which may be further substituted (e.g., with one or more substituents).

The term “alkyl” refers to a straight or branched hydrocarbon chain, containing the indicated number of carbon atoms. For example, C₁-C₁₂ alkyl indicates that the alkyl group may have from 1 to 12 (inclusive) carbon atoms. The term “alkylene” refers to a divalent alkyl, e.g., —CH₂—, —CH₂CH₂—, —CH₂CH₂CH₂— or —CH₂CH(CH₃) CH₂—. An alkyl or alkylene may be optionally substituted.

The term “alkenyl” refers to a straight or branched hydrocarbon chain having one or more double bonds. Examples of alkenyl groups include, but are not limited to, allyl, propenyl, 2-butenyl, 3-hexenyl and 3-octenyl groups. One of the double bond carbons may optionally be the point of attachment of the alkenyl substituent. The term “alkenylene” refers to a divalent alkenyl, e.g., —CH═CH—, —CH═CH₂CH₂—, or —CH═C═CH—. An alkenyl or alkenylene may be optionally substituted.

The term “alkynyl” refers to a straight or branched hydrocarbon chain having one or more triple bonds. Examples of alkynyl groups include, but are not limited to, ethynyl, propargyl, and 3-hexynyl. One of the triple bond carbons may optionally be the point of attachment of the alkynyl substituent. The term “alkynylene” refers to a divalent alkynyl, e.g., —CC— or —CCCH₂—. An alkynyl or alkynylene may be optionally substituted.

The term “amino” refers to a group of the formula —NR¹R², wherein R¹ and R² are each independently selected from, for example, hydrogen, alkyl, cycloalkyl, heterocyclyl, aryl and heteroaryl, or R¹ and R², together with the nitrogen to which they are attached, may form a ring structure. Examples of amino groups include, but are not limited to, —NH₂, alkylamino groups such as —NHCH₃, —NHCH₂CH₃ and —NHCH(CH₃)₂, dialkylamino groups such as —N(CH₃)₂ and —N(CH₂CH₃)₂, and arylamino groups such as —NHPh. Examples of cyclic amino groups include, but are not limited to, aziridinyl, azetidinyl, pyrrolidinyl, piperidino, piperazinyl, perhydrodiazepinyl, morpholino, and thiomorpholino. The groups R¹ and R² may be optionally substituted.

The term “aryl” refers to an aromatic monocyclic, bicyclic, or tricyclic hydrocarbon ring system, wherein any ring atom capable of substitution can be substituted (e.g., with one or more substituents). Examples of aryl moieties include, but are not limited to, phenyl, naphthyl, and anthracenyl.

The term “arylalkyl” refers to an alkyl moiety in which an alkyl hydrogen atom is replaced with an aryl group. Arylalkyl includes groups in which more than one hydrogen atom has been replaced with an aryl group. Examples of arylalkyl groups include benzyl, 2-phenylethyl, 3-phenylpropyl, 9-fluorenyl, benzhydryl, and trityl groups.

The term “cycloalkyl” as used herein refers to nonaromatic, saturated or partially unsaturated cyclic, bicyclic, tricyclic or polycyclic hydrocarbon groups having 3 to 12 carbons. Any ring atom can be substituted (e.g., with one or more substituents). Cycloalkyl groups can contain fused rings. Fused rings are rings that share one or more common carbon atoms. Examples of cycloalkyl groups include, but are not limited to, cyclopropyl, cyclobutyl, cyclopentyl, cyclohexyl, cyclohexenyl, cyclohexadienyl, methylcyclohexyl, adamantyl, norbornyl and norbornenyl.

The term “halo” or “halogen” as used herein refers to any radical of fluorine, chlorine, bromine, or iodine.

The term “haloalkyl” as used herein refers to an alkyl in which one or more hydrogen atoms are replaced with a halogen, and includes alkyl moieties in which all hydrogens have been replaced with halogens (e.g., perfluoroalkyl such as CF₃).

The term “heteroaryl” as used herein refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms independently selected from O, N, S, P and Si (e.g., carbon atoms and 1-3, 1-6, or 1-9 heteroatoms independently selected from O, N, S, P and Si if monocyclic, bicyclic, or tricyclic, respectively). Any ring atom can be substituted (e.g., with one or more substituents).

Heteroaryl groups can contain fused rings, which are rings that share one or more common atoms. Examples of heteroaryl groups include, but are not limited to, radicals of pyridine, pyrimidine, pyrazine, pyridazine, pyrrole, imidazole, pyrazole, oxazole, isoxazole, furan, thiazole, isothiazole, thiophene, quinoline, isoquinoline, quinoxaline, quinazoline, cinnoline, indole, isoindole, indolizine, indazole, benzimidazole, phthalazine, pteridine, carbazole, carboline, phenanthridine, acridine, phenanthroline, phenazine, naphthyridines and purines.

The term “heterocyclyl” as used herein refers to a nonaromatic, saturated or partially unsaturated 3-10 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, S, Si and P (e.g., carbon atoms and 1-3, 1-6, or 1-9 heteroatoms of O, N, S, Si and P if monocyclic, bicyclic, or tricyclic, respectively). Any ring atom can be substituted (e.g., with one or more substituents).

Heterocyclyl groups can contain fused rings, which are rings that share one or more common atoms. Examples of heterocyclyl groups include, but are not limited to, radicals of tetrahydrofuran, tetrahydrothiophene, tetrahydropyran, piperidine, piperazine, morpholine, pyrroline, pyrimidine, pyrrolidine, indoline, tetrahydropyridine, dihydropyran, thianthrene, pyran, benzopyran, xanthene, phenoxathiin, phenothiazine, furazan, lactones, lactams such as azetidinones and pyrrolidinones, sultams, sultones, and the like.

The term “hydroxy” refers to an —OH functional group. The term “alkoxy” refers to an —Oalkyl functional group.

The term “aryloxy” refers to an —O-aryl group.

The term “oxo” refers to an oxygen atom, which forms a carbonyl when attached to carbon, an N-oxide when attached to nitrogen, and a sulfoxide or sulfone when attached to sulfur.

The term “mercapto” or “thiol” refers to an —SH radical. The term “thioalkoxy” or “thioether” refers to an —S-alkyl radical. The term “thioaryloxy” refers to an —S-aryl radical.

The term “substituents” refers to a group “substituted” on an alkyl, alkenyl, alkynyl, cycloalkyl, cycloalkylalkyl, heterocyclyl, heterocyclylalkyl, aryl, arylalkyl, heteroaryl or heteroarylalkyl group at any atom of that group. Any atom can be substituted. Suitable substituents include, without limitation: acyl, acylamido, acyloxy, alkoxy, alkyl, alkenyl, alkynyl, amido, amino, carboxy, cyano, ester, halo, hydroxy, imino, nitro, oxo (e.g., C═O), phosphonate, sulfinyl, sulfonyl, sulfonate, sulfonamino, sulfonamido, thioamido, thiol, thioxo (e.g., C═S), and ureido. In embodiments, substituents on a group are independently any one single, or any combination of the aforementioned substituents. In embodiments, a substituent may itself be substituted with any one of the above substituents.

The above substituents may be abbreviated herein, for example, the abbreviations Me, Et, Ph, Ac and Ts represent methyl, ethyl, phenyl, acetyl and tosyl (p-toluenesulfonyl), respectively. A more comprehensive list of the abbreviations used by organic chemists of ordinary skill in the art appears in the first issue of each volume of the Journal of Organic Chemistry; this list is typically presented in a table entitled Standard List of Abbreviations. The abbreviations contained in said list, and all abbreviations used by organic chemists of ordinary skill in the art, are hereby incorporated by reference.

For compounds described herein, groups and substituents thereof may be selected in accordance with permitted valence of the atoms and the substituents, such that the selections and substitutions result in a stable compound, e.g., which does not spontaneously undergo transformation such as by rearrangement, cyclization, elimination, etc.

Where substituent groups are specified by their conventional chemical formulae, written from left to right, they optionally encompass substituents resulting from writing the structure from right to left, e.g., —CH₂O-optionally also recites —OCH₂—.

In accordance with a convention used in the art, the group is used in structural formulas herein to depict the bond that is the point of attachment of the moiety or substituent to the core or backbone structure.

Described herein is a method for using ascorbic acid to deliver H₂O₂ to cancer cells and activate ROS-responsive prodrugs. Cancer cells exhibit higher H₂O₂ levels and lower catalase activity, activating ROS-responsive prodrugs and causing cancer cell death. In contrast, normal cells' lower H₂O₂ and higher catalase activity inhibit prodrug activation, enabling regular cell growth. Pretreating with ascorbic acid accumulates extracellular H₂O₂. Normal and cancer cells respond differently due to distinct H₂O₂ metabolization abilities. This enhances H₂O₂ availability for prodrug activation, improving cancer cell toxicity. The Peng group reported ROS-responsive prodrugs, FAN-NM-CH₃ being notably 10×- and 16×-toxic than chlorambucil and melphalan respectively, linked to higher H₂O₂ levels (~2 μM) activating it in MDA-MB-468 cancer cells.

The combination of ascorbic acid with the prodrug FAN-NM-CH₃ demonstrates enhanced toxicity leading to approximately 80% cell death in MDA-MB-468 cells, 50% in MCF7 cells, and 60% in U-87 MG cells, while sparing MCF 10A cells. This synergistic effect is equivalent to the toxicity achieved with a 10 μM, 7 μM, and 20 μM dose of FAN-NM-CH₃ alone in MDA-MB-468, MCF 7, and U-87 MG cancer cells, respectively. In terms of fold differences, the doses were 10-fold, 3-fold, and 4-fold lower than the doses of FAN-NM-CH₃ alone in MDA-MB-468, MCF 7, and U-87 MG cancer cells, respectively, to produce the same level of toxicity towards cancer cells. This combination approach reduces drug dosage while maintaining selectivity and efficacy.

### Cancer Combination Therapy

The disclosed compounds can be used as single agents or in combination with one or more other drugs in the treatment, prevention, control, amelioration or reduction of risk of the aforementioned diseases, disorders and conditions for which the compound or the other drugs have utility, where the combination of drugs together are safer or more effective than either drug alone. The other drug(s) can be administered by a route and in an amount commonly used therefor, contemporaneously or sequentially with a disclosed compound. When a disclosed compound is used contemporaneously with one or more other drugs, a pharmaceutical composition in unit dosage form containing such drugs and the disclosed compound may be used. However, the combination therapy can also be administered on overlapping schedules. It is also envisioned that the combination of one or more active ingredients and a disclosed compound can be more efficacious than either as a single agent. Thus, when used in combination with one or more other active ingredients, the disclosed compounds and the other active ingredients can be used in lower doses than when each is used singly.

The pharmaceutical compositions and methods of the present invention can further comprise other therapeutically active compounds as noted herein which are usually applied in the treatment of the above mentioned pathological conditions.

The above combinations include combinations of a disclosed compound not only with one other active compound, but also with two or more other active compounds. Likewise, disclosed compounds can be used in combination with other drugs that are used in the prevention, treatment, control, amelioration, or reduction of risk of the diseases or conditions for which disclosed compounds are useful. Such other drugs can be administered, by a route and in an amount commonly used therefor, contemporaneously, or sequentially with a compound of the present invention. When a compound of the present invention is used contemporaneously with one or more other drugs, a pharmaceutical composition containing such other drugs in addition to a disclosed compound is preferred. Accordingly, the pharmaceutical compositions include those that also contain one or more other active ingredients, in addition to a compound of the present invention.

The weight ratio of a disclosed compound to the second active ingredient can be varied and will depend upon the effective dose of each ingredient. Generally, an effective dose of each will be used. Combinations of a compound of the present invention and other active ingredients will generally also be within the aforementioned range, but in each case, an effective dose of each active ingredient should be used.

Accordingly, the disclosed compounds can be used alone or in combination with other agents which are known to be beneficial in the subject indications or other drugs that affect receptors or enzymes that either increase the efficacy, safety, convenience, or reduce unwanted side effects or toxicity of the disclosed compounds. The subject compound and the other agent can be co-administered, either in concomitant therapy or in a fixed combination.

A compound or composition described herein may be used in combination with other known therapies. Administered “in combination,” as used herein, means that two (or more) different treatments are delivered to the subject during the course of the subject's affliction with the disease or disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disease or disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

A compound described herein and at least one additional therapeutic agent can be administered simultaneously, in the same or in separate compositions, or sequentially. For sequential administration, a compound or composition described herein can be administered first, and the additional agent can be administered subsequently, or the order of administration can be reversed.

In some embodiments, a compound or composition described herein can be administered in combination with other therapeutic treatment modalities, including surgery, radiation, cryosurgery, and/or thermotherapy. Such combination therapies may advantageously utilize lower dosages of the administered agent and/or other chemotherapeutic agent, thus avoiding possible toxicities or complications associated with the various therapies. The phrase “radiation” includes, but is not limited to, external-beam therapy which involves three-dimensional, conformal radiation therapy where the field of radiation is designed to conform to the volume of tissue treated; interstitial-radiation therapy where seeds of radioactive compounds are implanted using ultrasound guidance; and a combination of external-beam therapy and interstitial-radiation therapy.

In some embodiments, a compound or composition described herein are administered with at least one additional therapeutic agent, such as a chemotherapeutic agent. In certain embodiments, a compound or composition described herein are administered in combination with one or more additional chemotherapeutic agents, e.g., with one or more chemotherapeutic agents described herein.

In some embodiments, a compound or composition described herein is administered in combination with one or more additional chemotherapeutic agents. Any chemotherapeutic agent can be used; exemplary chemotherapeutic agents are well-known to those skilled in the art. A compound or composition may also be administered in combination with other agents that may be administered to a subject in need thereof, such as hormones, steroids, antimicrobial agents, an agent or procedure to mitigate potential side effects from the agent compositions such as diarrhea, nausea and vomiting, an immunosuppressive agent, a CYP3A4 inhibitor, an antiemetic, and the like.

When formulating the pharmaceutical compositions described herein, the clinician may utilize preferred dosages as warranted by the condition of the subject being treated. For example, in one embodiment, a compound or composition described herein may be administered at a dosing schedule described herein, e.g., once every one, two, three, four, five or six weeks.

Also, in general, a compound or composition described herein, and an optional additional chemotherapeutic agent(s) do not have to be administered in the same pharmaceutical composition, and may, because of different physical and chemical characteristics, have to be administered by different routes. The determination of the mode of administration and the advisability of administration, where possible, in the same pharmaceutical composition, is well within the knowledge of the skilled clinician. The initial administration can be made according to established protocols known in the art, and then, based upon the observed effects, the dosage, modes of administration and times of administration can be modified by the skilled clinician.

The actual dosage of a compound or composition described herein and/or any additional chemotherapeutic agent employed may be varied depending upon the requirements of the subject and the severity of the condition being treated. Determination of the proper dosage for a particular situation is within the skill of the art. Generally, treatment is initiated with smaller dosages which are less than the optimum dose of the compound. Thereafter, the dosage is increased by small amounts until the optimum effect under the circumstances is reached.

In some embodiments, when a compound or composition described herein is administered in combination with one or more additional chemotherapeutic agents, the additional chemotherapeutic agent (or agents) is administered at a standard dose.

The particular choice of additional anti-proliferative cytotoxic agent(s) or radiation will depend upon the diagnosis of the attending physicians and their judgment of the condition of the subject and the appropriate treatment protocol.

If a compound or composition described herein and the additional chemotherapeutic agent(s) and/or radiation are not administered simultaneously or essentially simultaneously, then the initial order of administration of a compound or composition described herein, and the additional chemotherapeutic agent(s) and/or radiation, may be varied. Thus, for example a compound or composition described herein may be administered first followed by the administration of the additional chemotherapeutic agent(s) and/or radiation; or the additional chemotherapeutic agent(s) and/or radiation may be administered first followed by the administration of a compound or composition described herein. This alternate administration may be repeated during a single treatment protocol. The determination of the order of administration, and the number of repetitions of administration of each therapeutic agent during a treatment protocol, is well within the knowledge of the skilled physician after evaluation of the disease being treated and the condition of the subject.

Thus, in accordance with experience and knowledge, the practicing physician can modify each protocol for the administration of a component (a compound or composition described herein, anti-neoplastic agent(s), or radiation) of the treatment according to the individual subject's needs, as the treatment proceeds.

The attending clinician, in judging whether treatment is effective at the dosage administered, will consider the general well-being of the subject as well as more definite signs such as relief of disease-related symptoms, inhibition of tumor growth, actual shrinkage of the tumor, or inhibition of metastasis. Size of the tumor can be measured by standard methods such as radiological studies, e.g., CAT or MRI scan, and successive measurements can be used to judge whether or not growth of the tumor has been retarded or even reversed. Relief of disease related symptoms such as pain, and improvement in overall condition can also be used to help judge effectiveness of treatment.

### Pharmaceutical Salts

The disclosed compounds may exist as pharmaceutically acceptable salts. The term “pharmaceutically acceptable salt” refers to salts or zwitterions of the compounds which are water or oil-soluble or dispersible, suitable for treatment of disorders without undue toxicity, irritation, and allergic response, commensurate with a reasonable benefit/risk ratio and effective for their intended use. The salts may be prepared during the final isolation and purification of the compounds or separately by reacting an amino group of the compounds with a suitable acid. For example, a compound may be dissolved in a suitable solvent, such as but not limited to methanol and water and treated with at least one equivalent of an acid, like hydrochloric acid. The resulting salt may precipitate out and be isolated by filtration and dried under reduced pressure. Alternatively, the solvent and excess acid may be removed under reduced pressure to provide a salt. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, thrichloroacetate, trifluoroacetate, glutamate, para-toluenesulfonate, undecanoate, hydrochloric, hydrobromic, sulfuric, phosphoric and the like. The amino groups of the compounds may also be quaternized with alkyl chlorides, bromides, or iodides such as methyl, ethyl, propyl, isopropyl, butyl, lauryl, myristyl, stearyl and the like.

Basic addition salts may be prepared during the final isolation and purification of the disclosed compounds by reaction of a carboxyl group with a suitable base such as the hydroxide, carbonate, or bicarbonate of a metal cation such as lithium, sodium, potassium, calcium, magnesium, or aluminum, or an organic primary, secondary, or tertiary amine. Quaternary amine salts can be prepared, such as those derived from methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine and N,N′-dibenzylethylenediamine, ethylenediamine, ethanolamine, diethanolamine, piperidine, piperazine, and the like.

### Formulations

The disclosed compounds may be incorporated into pharmaceutical compositions suitable for administration to a subject (such as a patient, which may be a human or non-human). The disclosed compounds may also be provided as formulations.

The pharmaceutical compositions and formulations may include a “therapeutically effective amount” or a “prophylactically effective amount” of the agent. A “therapeutically effective amount” refers to an amount effective, at dosages and for periods of time necessary, to achieve the desired therapeutic result. A therapeutically effective amount of the composition may be determined by a person skilled in the art and may vary according to factors such as the disease state, age, sex, and weight of the individual, and the ability of the composition to elicit a desired response in the individual. A therapeutically effective amount is also one in which any toxic or detrimental effects of a compound of the invention (e.g., a compound of formula (I)) are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

While a compound described herein may be administered alone in the methods described herein, it may also be presented as one or more pharmaceutical compositions (e.g., formulations). A compound described herein may be formulated with one or more pharmaceutically acceptable carriers, adjuvants, excipients, diluents, fillers, buffers, stabilizers, preservatives, lubricants, or other materials well known to those skilled in the art and optionally other therapeutic or prophylactic agents.

Accordingly, the methods described herein include administration of one or more pharmaceutical compositions, as discussed herein, in which a compound described herein is admixed together with one or more pharmaceutically acceptable carriers, excipients, buffers, adjuvants, stabilizers, or other materials, as described herein.

Suitable carriers, excipients, etc. can be found in standard pharmaceutical texts, for example, Remington's Pharmaceutical Sciences, 18th edition, Mack Publishing Company, Easton, Pa., 1990.

The formulations may conveniently be presented in unit dosage form and may be prepared by any methods known in the art of pharmacy. Such methods include the step of bringing into association the active compound(s) with the carrier which constitutes one or more accessory ingredients. In general, the formulations are prepared by uniformly and intimately bringing into association the active compound with liquid carriers or finely divided solid carriers or both, and then if necessary shaping the product.

Formulations may be in the form of liquids, solutions, suspensions, emulsions, elixirs, syrups, tablets, lozenges, granules, powders, capsules, cachets, pills, ampoules, suppositories, pessaries, ointments, gels, pastes, creams, sprays, mists, foams, lotions, oils, boluses, electuaries, or aerosols.

Formulations suitable for oral administration (e.g., by ingestion) may be presented as discrete units such as capsules, cachets or tablets, each containing a predetermined amount of the active compound; as a powder or granules; as a solution or suspension in an aqueous or nonaqueous liquid; or as an oil-in-water liquid emulsion or a water-in-oil liquid emulsion; as a bolus; as an electuary; or as a paste.

A tablet may be made by conventional means, e.g., compression or molding, optionally with one or more accessory ingredients. Compressed tablets may be prepared by compressing in a suitable machine the active compound in a free-flowing form such as a powder or granules, optionally mixed with one or more binders (e.g., povidone, gelatin, acacia, sorbitol, tragacanth, hydroxypropylmethyl cellulose); fillers or diluents (e.g., lactose, microcrystalline cellulose, calcium hydrogen phosphate); lubricants (e.g., magnesium stearate, talc, silica); disintegrants (e.g., sodium starch glycolate, cross-linked povidone, cross-linked sodium carboxymethyl cellulose); surface-active or dispersing or wetting agents (e.g., sodium lauryl sulfate); and preservatives (e.g., methyl p-hydroxybenzoate, propyl p-hydroxybenzoate, sorbic acid). Molded tablets may be made by molding in a suitable machine a mixture of the powdered compound moistened with an inert liquid diluent. The tablets may optionally be coated or scored and may be formulated so as to provide slow or controlled release of the active compound therein using, for example, hydroxypropylmethyl cellulose in varying proportions to provide the desired release profile. Tablets may optionally be provided with an enteric coating, to provide release in parts of the gut other than the stomach.

Formulations suitable for parenteral administration (e.g., by injection, including cutaneous, subcutaneous, intramuscular, intravenous and intradermal), include aqueous and nonaqueous isotonic, pyrogen-free, sterile injection solutions which may contain anti-oxidants, buffers, preservatives, stabilizers, bacteriostats, and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. Examples of suitable isotonic vehicles for use in such formulations include Sodium Chloride Injection, Ringer's Solution, or Lactated Ringer's Injection. The formulations may be presented in unit-dose or multi-dose sealed containers, for example, ampoules and vials, and may be stored in a freeze-dried (lyophilized) condition requiring only the addition of the sterile liquid carrier, for example water for injections, immediately prior to use.

Extemporaneous injection solutions and suspensions may be prepared from sterile powders, granules, and tablets. Formulations may be in the form of liposomes or other microparticulate systems which are designed to target the active compound to blood components or one or more organs.

Formulations suitable for topical administration (e.g., transdermal, intranasal, ocular, buccal, and sublingual) may be formulated as an ointment, cream, suspension, lotion, powder, solution, past, gel, spray, aerosol, or oil. Alternatively, a formulation may comprise a patch or a dressing such as a bandage or adhesive plaster impregnated with active compounds and optionally one or more excipients or diluents.

Formulations suitable for topical administration in the mouth include lozenges comprising the active compound in a flavored basis, usually sucrose and acacia or tragacanth; pastilles comprising the active compound in an inert basis such as gelatin and glycerin, or sucrose and acacia; and mouthwashes comprising the active compound in a suitable liquid carrier.

Formulations suitable for topical administration to the eye also include eye drops wherein the active compound is dissolved or suspended in a suitable carrier, especially an aqueous solvent for the active compound.

Formulations suitable for nasal administration, wherein the carrier is a solid, include a coarse powder having a particle size, for example, in the range of about 20 to about 500 microns which is administered in the manner in which snuff is taken, i.e., by rapid inhalation through the nasal passage from a container of the powder held close up to the nose. Suitable formulations wherein the carrier is a liquid for administration as, for example, nasal spray, nasal drops, or by aerosol administration by nebulizer, include aqueous or oily solutions of the active compound.

Formulations suitable for administration by inhalation include those presented as an aerosol spray from a pressurized pack, with the use of a suitable propellant, such as dichlorodifluoromethane, trichlorofluoromethane, dichoro-tetrafluoroethane, carbon dioxide, or other suitable gases. Further formulations suitable for inhalation include those presented as a nebulizer.

Formulations suitable for topical administration via the skin include ointments, creams, and emulsions. When formulated in an ointment, the active compound may optionally be employed with either a paraffinic or a water-miscible ointment base. Alternatively, the active compounds may be formulated in a cream with an oil-in-water cream base. If desired, the aqueous phase of the cream base may include, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof. The topical formulations may desirably include a compound which enhances absorption or penetration of the active compound through the skin or other affected areas. Examples of such dermal penetration enhancers include dimethylsulfoxide and related analogues.

When formulated as a topical emulsion, the oily phase may optionally comprise merely an emulsifier (otherwise known as an emulgent), or it may comprises a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat.

Together, the emulsifier(s) with or without stabilizer(s) make up the so-called emulsifying wax, and the wax together with the oil and/or fat make up the so-called emulsifying ointment base which forms the oily dispersed phase of the cream formulations.

Suitable emulgents and emulsion stabilizers include Tween 60, Span 80, cetostearyl alcohol, myristyl alcohol, glyceryl monostearate and sodium lauryl sulfate. The choice of suitable oils or fats for the formulation is based on achieving the desired cosmetic properties, since the solubility of the active compound in most oils likely to be used in pharmaceutical emulsion formulations may be very low. Thus the cream should preferably be a non-greasy, non-staining and washable product with suitable consistency to avoid leakage from tubes or other containers. Straight or branched chain, mono- or dibasic alkyl esters such as diisoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters.

These may be used alone or in combination depending on the properties required. Alternatively, high melting point lipids such as white soft paraffin and/or liquid paraffin or other mineral oils can be used.

Formulations suitable for rectal administration may be presented as a suppository with a suitable base comprising, for example, cocoa butter or a salicylate.

Formulations suitable for vaginal administration may be presented as pessaries, tampons, creams, gels, pastes, foams or spray formulations containing in addition to the active compound, such carriers as are known in the art to be appropriate.

### Pharmaceutical Compositions and Formulations

Thus, the compounds and their pharmaceutically acceptable salts may be formulated for administration by, for example, solid dosing, eye drop, in a topical oil-based formulation, injection, inhalation (either through the mouth or the nose), implants, or oral, buccal, parenteral, or rectal administration. Techniques and formulations may generally be found in “Remington's Pharmaceutical Sciences,” (Meade Publishing Co., Easton, Pa.). Therapeutic compositions must typically be sterile and stable under the conditions of manufacture and storage.

The route by which the disclosed compounds are administered, and the form of the composition will dictate the type of carrier to be used. The composition may be in a variety of forms, suitable, for example, for systemic administration (e.g., oral, rectal, nasal, sublingual, buccal, implants, or parenteral) or topical administration (e.g., dermal, pulmonary, nasal, aural, ocular, liposome delivery systems, or iontophoresis).

Carriers for systemic administration typically include at least one of diluents, lubricants, binders, disintegrants, colorants, flavors, sweeteners, antioxidants, preservatives, glidants, solvents, suspending agents, wetting agents, surfactants, combinations thereof, and others. All carriers are optional in the compositions.

Suitable diluents include sugars such as glucose, lactose, dextrose, and sucrose; diols such as propylene glycol; calcium carbonate; sodium carbonate; sugar alcohols, such as glycerin; mannitol; and sorbitol. The amount of diluent(s) in a systemic or topical composition is typically about 50 to about 90%.

Suitable lubricants include silica, talc, stearic acid and its magnesium salts and calcium salts, calcium sulfate; and liquid lubricants such as polyethylene glycol and vegetable oils such as peanut oil, cottonseed oil, sesame oil, olive oil, corn oil and oil of theobroma. The amount of lubricant(s) in a systemic or topical composition is typically about 5 to about 10%.

Suitable binders include polyvinyl pyrrolidone; magnesium aluminum silicate; starches such as corn starch and potato starch; gelatin; tragacanth; and cellulose and its derivatives, such as sodium carboxymethylcellulose, ethyl cellulose, methylcellulose, microcrystalline cellulose, and sodium carboxymethylcellulose. The amount of binder(s) in a systemic composition is typically about 5 to about 50%.

Suitable disintegrants include agar, alginic acid and the sodium salt thereof, effervescent mixtures, croscarmellose, crospovidone, sodium carboxymethyl starch, sodium starch glycolate, clays, and ion exchange resins. The amount of disintegrant(s) in a systemic or topical composition is typically about 0.1 to about 10%.

Suitable colorants include a colorant such as an FD&C dye. When used, the amount of colorant in a systemic or topical composition is typically about 0.005 to about 0.1%.

Suitable flavors include menthol, peppermint, and fruit flavors. The amount of flavor(s), when used, in a systemic or topical composition is typically about 0.1 to about 1.0%.

Suitable sweeteners include aspartame and saccharin. The amount of sweetener(s) in a systemic or topical composition is typically about 0.001 to about 1%.

Suitable antioxidants include butylated hydroxyanisole (“BHA”), butylated hydroxytoluene (“BHT”), and vitamin E. The amount of antioxidant(s) in a systemic or topical composition is typically about 0.1 to about 5%.

Suitable preservatives include benzalkonium chloride, methyl paraben and sodium benzoate. The amount of preservative(s) in a systemic or topical composition is typically about 0.01 to about 5%.

Suitable glidants include silicon dioxide. The amount of glidant(s) in a systemic or topical composition is typically about 1 to about 5%.

Suitable solvents include water, isotonic saline, ethyl oleate, glycerine, hydroxylated castor oils, alcohols such as ethanol, and phosphate buffer solutions. The amount of solvent(s) in a systemic or topical composition is typically from about 0 to about 100%.

Suitable suspending agents include AVICEL RC-591 (from FMC Corporation of Philadelphia, PA) and sodium alginate. The amount of suspending agent(s) in a systemic or topical composition is typically about 1 to about 8%.

Suitable surfactants include lecithin, Polysorbate 80, and sodium lauryl sulfate, and the TWEENS from Atlas Powder Company of Wilmington, Delaware. Suitable surfactants include those disclosed in the C.T.F.A. Cosmetic Ingredient Handbook, 1992, pp. 587-592; Remington's Pharmaceutical Sciences, 15th Ed. 1975, pp. 335-337; and Mccutcheon's Volume 1, Emulsifiers & Detergents, 1994, North American Edition, pp. 236-239. The amount of surfactant(s) in the systemic or topical composition is typically about 0.1% to about 5%.

Although the amounts of components in the systemic compositions may vary depending on the type of systemic composition prepared, in general, systemic compositions include 0.01% to 50% of an active compound (e.g., a compound of formula (I)) and 50% to 99.99% of one or more carriers. Compositions for parenteral administration typically include 0.1% to 10% of actives and 90% to 99.9% of a carrier including a diluent and a solvent.

Compositions for oral administration can have various dosage forms. For example, solid forms include tablets, capsules, granules, and bulk powders. These oral dosage forms include a safe and effective amount, usually at least about 5%, and more particularly from about 25% to about 50% of actives. The oral dosage compositions include about 50% to about 95% of carriers, and more particularly, from about 50% to about 75%.

Tablets can be compressed, tablet triturates, enteric-coated, sugar-coated, film-coated, or multiple-compressed. Tablets typically include an active component, and a carrier comprising ingredients selected from diluents, lubricants, binders, disintegrants, colorants, flavors, sweeteners, glidants, and combinations thereof. Specific diluents include calcium carbonate, sodium carbonate, mannitol, lactose and cellulose. Specific binders include starch, gelatin, and sucrose. Specific disintegrants include alginic acid and croscarmellose. Specific lubricants include magnesium stearate, stearic acid, and talc. Specific colorants are the FD&C dyes, which can be added for appearance. Chewable tablets preferably contain sweeteners such as aspartame and saccharin, or flavors such as menthol, peppermint, fruit flavors, or a combination thereof.

Capsules (including implants, time release and sustained release formulations) typically include an active compound (e.g., a compound of formula (I)), and a carrier including one or more diluents disclosed above in a capsule comprising gelatin. Granules typically comprise a disclosed compound, and preferably glidants such as silicon dioxide to improve flow characteristics. Implants can be of the biodegradable or the non-biodegradable type.

The selection of ingredients in the carrier for oral compositions depends on secondary considerations like taste, cost, and shelf stability, which are not critical for the purposes of this invention.

Solid compositions may be coated by conventional methods, typically with pH or time-dependent coatings, such that a disclosed compound is released in the gastrointestinal tract in the vicinity of the desired application, or at various points and times to extend the desired action. The coatings typically include one or more components selected from the group consisting of cellulose acetate phthalate, polyvinyl acetate phthalate, hydroxypropyl methyl cellulose phthalate, ethyl cellulose, EUDRAGIT® coatings (available from Evonik Industries of Essen, Germany), waxes and shellac.

Compositions for oral administration can have liquid forms. For example, suitable liquid forms include aqueous solutions, emulsions, suspensions, solutions reconstituted from non-effervescent granules, suspensions reconstituted from non-effervescent granules, effervescent preparations reconstituted from effervescent granules, elixirs, tinctures, syrups, and the like. Liquid orally administered compositions typically include a disclosed compound and a carrier, namely, a carrier selected from diluents, colorants, flavors, sweeteners, preservatives, solvents, suspending agents, and surfactants. Peroral liquid compositions preferably include one or more ingredients selected from colorants, flavors, and sweeteners.

Other compositions useful for attaining systemic delivery of the subject compounds include sublingual, buccal and nasal dosage forms. Such compositions typically include one or more of soluble filler substances such as diluents including sucrose, sorbitol and mannitol; and binders such as acacia, microcrystalline cellulose, carboxymethyl cellulose, and hydroxypropyl methylcellulose. Such compositions may further include lubricants, colorants, flavors, sweeteners, antioxidants, and glidants.

The disclosed compounds can be topically administered. Topical compositions that can be applied locally to the skin may be in any form including solids, solutions, oils, creams, ointments, gels, lotions, shampoos, leave-on and rinse-out hair conditioners, milks, cleansers, moisturizers, sprays, skin patches, and the like. Topical compositions include: a disclosed compound (e.g., a compound of formula (I)), and a carrier. The carrier of the topical composition preferably aids penetration of the compounds into the skin. The carrier may further include one or more optional components.

The amount of the carrier employed in conjunction with a disclosed compound is sufficient to provide a practical quantity of composition for administration per unit dose of the compound. Techniques and compositions for making dosage forms useful in the methods of this invention are described in the following references: Modern Pharmaceutics, Chapters 9 and 10, Banker & Rhodes, eds. (1979); Lieberman et al., Pharmaceutical Dosage Forms: Tablets (1981); and Ansel, Introduction to Pharmaceutical Dosage Forms, 2nd Ed., (1976).

A carrier may include a single ingredient or a combination of two or more ingredients. In the topical compositions, the carrier includes a topical carrier. Suitable topical carriers include one or more ingredients selected from phosphate buffered saline, isotonic water, deionized water, monofunctional alcohols, symmetrical alcohols, aloe vera gel, allantoin, glycerin, vitamin A and E oils, mineral oil, propylene glycol, PPG-2 myristyl propionate, dimethyl isosorbide, castor oil, combinations thereof, and the like. More particularly, carriers for skin applications include propylene glycol, dimethyl isosorbide, and water, and even more particularly, phosphate buffered saline, isotonic water, deionized water, monofunctional alcohols, and symmetrical alcohols.

The carrier of a topical composition may further include one or more ingredients selected from emollients, propellants, solvents, humectants, thickeners, powders, fragrances, pigments, and preservatives, all of which are optional.

Suitable emollients include stearyl alcohol, glyceryl monoricinoleate, glyceryl monostearate, propane-1,2-diol, butane-1,3-diol, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, cetyl palmitate, di-n-butyl sebacate, isopropyl myristate, isopropyl palmitate, isopropyl stearate, butyl stearate, polyethylene glycol, triethylene glycol, lanolin, sesame oil, coconut oil, arachis oil, castor oil, acetylated lanolin alcohols, petroleum, mineral oil, butyl myristate, isostearic acid, palmitic acid, isopropyl linoleate, lauryl lactate, myristyl lactate, decyl oleate, myristyl myristate, and combinations thereof. Specific emollients for skin include stearyl alcohol and polydimethylsiloxane. The amount of emollient(s) in a skin-based topical composition is typically about 5% to about 95%.

Suitable propellants include propane, butane, isobutane, dimethyl ether, carbon dioxide, nitrous oxide, and combinations thereof. The amount of propellant(s) in a topical composition is typically about 0% to about 95%.

Suitable solvents include water, ethyl alcohol, methylene chloride, isopropanol, castor oil, ethylene glycol monoethyl ether, diethylene glycol monobutyl ether, diethylene glycol monoethyl ether, dimethylsulfoxide, dimethyl formamide, tetrahydrofuran, and combinations thereof. Specific solvents include ethyl alcohol and homotopic alcohols. The amount of solvent(s) in a topical composition is typically about 0% to about 95%.

Suitable humectants include glycerin, sorbitol, sodium 2-pyrrolidone-5-carboxylate, soluble collagen, dibutyl phthalate, gelatin, and combinations thereof. Specific humectants include glycerin. The amount of humectant(s) in a topical composition is typically 0% to 95%.

The amount of thickener(s) in a topical composition is typically about 0% to about 95%.

Suitable powders include beta-cyclodextrins, hydroxypropyl cyclodextrins, chalk, talc, fullers earth, kaolin, starch, gums, colloidal silicon dioxide, sodium polyacrylate, tetra alkyl ammonium smectites, trialkyl aryl ammonium smectites, chemically-modified magnesium aluminum silicate, organically-modified montmorillonite clay, hydrated aluminum silicate, fumed silica, carboxyvinyl polymer, sodium carboxymethyl cellulose, ethylene glycol monostearate, and combinations thereof. The amount of powder(s) in a topical composition is typically 0% to 95%.

The amount of fragrance in a topical composition is typically about 0% to about 0.5%, particularly, about 0.001% to about 0.1%.

Suitable pH adjusting additives include HCl or NaOH in amounts sufficient to adjust the pH of a topical pharmaceutical composition.

### Dosages

It will be appreciated that appropriate dosages of the active compounds and compositions comprising the active compounds, can vary from patient to patient. Determining the optimal dosage will generally involve the balancing of the level of therapeutic benefit against any risk or deleterious side effects of the treatments described herein. The selected dosage level will depend on a variety of factors including, but not limited to, the activity of the particular compound, the route of administration, the time of administration, the rate of excretion of the compound, the duration of the treatment, other drugs, compounds, and/or materials used in combination, and the age, sex, weight, condition, general health, and prior medical history of the patient. The amount of compound and route of administration will ultimately be at the discretion of the physician, although generally the dosage will be to achieve local concentrations at the site of action which achieve the desired effect without causing substantial harmful or deleterious side-effects.

Administration in vivo can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician.

In general, a suitable dose of the active compound may be in the range of about 100 g to about 250 mg per kilogram body weight of the subject per day.

In one embodiment the dose of an antineoplastic agent comprises from about 0.001 to about 200 mg/kg. In one aspect, the dose comprises about 0.001 mg/kg, 0.002 mg/kg, 0.003 mg/kg, 0.004 mg/kg, 0.005 mg/kg, 0.006 mg/kg, 0.007 mg/kg, 0.008 mg/kg, 0.009 mg/kg, 0.01 to about 20 mg/kg, 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg, 0.1 mg/kg, 0.11 mg/kg, 0.12 mg/kg, 0.13 mg/kg, 0.14 mg/kg, 0.15 mg/kg, 0.16 mg/kg, 0.17 mg/kg, 0.18 mg/kg, 0.19 mg/kg, 0.2 mg/kg, 0.21 mg/kg, 0.22 mg/kg, 0.23 mg/kg, 0.24 mg/kg, 0.25 mg/kg, 0.26 mg/kg, 0.27 mg/kg, 0.28 mg/kg, 0.29 mg/kg, 0.3 mg/kg, 0.31 mg/kg, 0.32 mg/kg, 0.33 mg/kg, 0.34 mg/kg, 0.35 mg/kg, 0.36 mg/kg, 0.37 mg/kg, 0.38 mg/kg, 0.39 mg/kg, 0.4 mg/kg, 0.41 mg/kg, 0.42 mg/kg, 0.43 mg/kg, 0.44 mg/kg, 0.45 mg/kg, 0.46 mg/kg, 0.47 mg/kg, 0.48 mg/kg, 0.49 mg/kg, 0.5 mg/kg, 0.6 mg/kg, 0.7 mg/kg, 0.8 mg/kg, 0.9 mg/kg, 1 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3 mg/kg, 3.1 mg/kg, 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, 5 mg/kg, 5.5 mg/kg, 6 mg/kg, 5.3 mg/kg, 5.4 mg/kg, 5.5 mg/kg, 5.6 mg/kg, 5.7 mg/kg, 5.8 mg/kg, 5.9 mg/kg, 6 mg/kg, 6.1 mg/kg, 6.2 mg/kg, 6.3 mg/kg, 6.4 mg/kg, 6.5 mg/kg, 6.6 mg/kg, 6.7 mg/kg, 6.8 mg/kg, 6.9 mg/kg, 7 mg/kg, 7.1 mg/kg, 7.2 mg/kg, 7.3 mg/kg, 7.4 mg/kg, 7.5 mg/kg, 7.6 mg/kg, 7.7 mg/kg, 7.8 mg/kg, 7.9 mg/kg, 8 mg/kg, 8.1 mg/kg, 8.2 mg/kg, 8.3 mg/kg, 8.4 mg/kg, 8.5 mg/kg, 8.6 mg/kg, 8.7 mg/kg, 8.8 mg/kg, 8.9 mg/kg, 9 mg/kg, 9.1 mg/kg, 9.2 mg/kg, 9.3 mg/kg, 9.4 mg/kg, 9.5 mg/kg, 9.6 mg/kg, 9.7 mg/kg, 9.8 mg/kg, 9.9 mg/kg, 10 mg/kg, 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 15.5 mg/kg, 16 mg/kg, 16.5 mg/kg, 17 mg/kg, 17.5 mg/kg, 18 mg/kg, 18.5 mg/kg, 19 mg/kg, 19.5 mg/kg, 20 mg/kg, 40 mg/kg, 60 mg/kg, 80 mg/kg, 100 mg/kg, 120 mg/kg, 140 mg/kg, 160 mg/kg, 180 mg/kg, 200 mg/kg or greater.

In another aspect, the dose of an antineoplastic agent comprises about 0.001-0.1 mg/kg, 0.002-0.1 mg/kg, 0.003-0.1 mg/kg, 0.004-0.1 mg/kg, 0.005-0.1 mg/kg, 0.006-0.1 mg/kg, 0.007-0.1 mg/kg, 0.008-0.1 mg/kg, 0.009-0.1 mg/kg, 0.001-0.5 mg/kg, 0.001-1 mg/kg, 0.001-1.5 mg/kg, 0.001-2 mg/kg, 0.001-2.5 mg/kg, 0.001-3 mg/kg, 0.001-3.5 mg/kg, 0.001-4 mg/kg, 0.001-4.5 mg/kg, 0.001-5 mg/kg, 0.001-10 mg/kg, 0.001-15 mg/kg, 0.001-20 mg/kg, 0.001-25 mg/kg, 0.001-50 mg/kg, 0.001-75 mg/kg, 0.001-100 mg/kg, 0.001-125 mg/kg, 0.001-150 mg/kg, 0.001-175 mg/kg, 0.001-200 mg/kg, 0.01-10 mg/kg, 0.01-15 mg/kg, 0.01-20 mg/kg, 0.01-0.1 mg/kg, 0.01-0.2 mg/kg, 0.01-0.3 mg/kg, 0.01-0.4 mg/kg, 0.01-0.5 mg/kg, 0.01-0.6 mg/kg, 0.01-0.7 mg/kg, 0.01-0.8 mg/kg, 0.01-0.9 mg/kg, 0.01-1 mg/kg, 0.01-5 mg/kg, 0.01-10 mg/kg, 0.01-15 mg/kg, 0.01-20 mg/kg, 0.01-25 mg/kg, 0.01-50 mg/kg, 0.01-75 mg/kg, 0.01-100 mg/kg, 0.01-125 mg/kg, 0.01-150 mg/kg, 0.01-175 mg/kg, 0.01-200 mg/kg, 0.1-1 mg/kg, 0.1-1.5 mg/kg, 0.1-2 mg/kg, 0.1-2.5 mg/kg, 0.1-3 mg/kg, 0.1-3.5 mg/kg, 0.1-4 mg/kg, 0.1-4.5 mg/kg, 0.1-5 mg/kg, 0.1-10 mg/kg, 0.1-15 mg/kg, 0.1-20 mg/kg, 0.1-25 mg/kg, 0.1-50 mg/kg, 0.1-75 mg/kg, 0.1-100 mg/kg, 0.1-125 mg/kg, 0.1-150 mg/kg, 0.1-175 mg/kg, 0.1-200 mg/kg, 1-5 mg/kg, 1-5.5 mg/kg, 1-6 mg/kg, 1-6.5 mg/kg, 1-7 mg/kg, 1-7.5 mg/kg, 1-8 mg/kg, 1-8.5 mg/kg, 1-9 mg/kg, 1-9.5 mg/kg, 1-10 mg/kg, 1-10.5 mg/kg, 1-11 mg/kg, 1-11.5 mg/kg, 1-12 mg/kg, 1-12.5 mg/kg, 1-13 mg/kg, 1-13.5 mg/kg, 1-14 mg/kg, 1-14.5 mg/kg, 1-15 mg/kg, 1-15.5 mg/kg, 1-16 mg/kg, 1-16.5 mg/kg, 1-17 mg/kg, 1-17.5 mg/kg, 1-18 mg/kg, 1-18.5 mg/kg, 1-19 mg/kg, 1-19.5 mg/kg, 1-20 mg/kg, 1-25 mg/kg, 1-50 mg/kg, 1-75 mg/kg, 1-100 mg/kg, 1-125 mg/kg, 1-150 mg/kg, 1-175 mg/kg, 1-200 mg/kg, 5-10 mg/kg, 5-10.5 mg/kg, 5-11 mg/kg, 5-11.5 mg/kg, 5-12 mg/kg, 5-12.5 mg/kg, 5-13 mg/kg, 5-13.5 mg/kg, 5-14 mg/kg, 5-14.5 mg/kg, 5-15 mg/kg, 5-15.5 mg/kg, 5-16 mg/kg, 5-16.5 mg/kg, 5-17 mg/kg, 5-17.5 mg/kg, 5-18 mg/kg, 5-18.5 mg/kg, 5-19 mg/kg, 5-19.5 mg/kg, or 5-20 mg/kg, 5-25 mg/kg, 5-50 mg/kg, 5-75 mg/kg, 5-100 mg/kg, 5-125 mg/kg, 5-150 mg/kg, 5-175 mg/kg, 5-200 mg/kg, 10-25 mg/kg, 10-50 mg/kg, 10-75 mg/kg, 10-100 mg/kg, 10-125 mg/kg, 10-150 mg/kg, 10-175 mg/kg, 10-200 mg/kg, including all endpoints, integers and subranges within the disclosed ranges.

In one embodiment the dose of a pro-oxidant, salt thereof, or prodrug thereof, comprises from about 0.01 to about 20 mg/kg. In one aspect, the dose comprises about 1 mg/kg, 5 mg/kg, 10 mg/kg, 20 mg/kg, 30 mg/kg, 40 mg/kg, 50 mg/kg, 60 mg/kg, 70 mg/kg, 80 mg/kg, 90 mg/kg, 100 mg/kg, 110 mg/kg, 120 mg/kg, 130 mg/kg, 140 mg/kg, 150 mg/kg, 160 mg/kg, 170 mg/kg, 180 mg/kg, 190 mg/kg, 200 mg/kg, 210 mg/kg, 220 mg/kg, 230 mg/kg, 240 mg/kg, 250 mg/kg, 260 mg/kg, 270 mg/kg, 280 mg/kg, 290 mg/kg, 300 mg/kg, 310 mg/kg, 320 mg/kg, 330 mg/kg, 340 mg/kg, 350 mg/kg, 360 mg/kg, 370 mg/kg, 380 mg/kg, 390 mg/kg, 400 mg/kg, 410 mg/kg, 420 mg/kg, 430 mg/kg, 440 mg/kg, 450 mg/kg, 460 mg/kg, 470 mg/kg, 480 mg/kg, 490 mg/kg, 500 mg/kg, 500 mg/kg, 525 mg/kg, 550 mg/kg, 575 mg/kg, 600 mg/kg, 625 mg/kg, 650 mg/kg, 675 mg/kg, 700 mg/kg, 725 mg/kg, 750 mg/kg, 775 mg/kg, 800 mg/kg, 825 mg/kg, 850 mg/kg, 875 mg/kg, 900 mg/kg, 925 mg/kg, 950 mg/kg, 975 mg/kg, 1000 mg/kg, 1025 mg/kg, 1050 mg/kg, 1075 mg/kg, 1100 mg/kg, 1125 mg/kg, 1150 mg/kg, 1175 mg/kg, 1200 mg/kg, 1225 mg/kg, 1250 mg/kg, 1275 mg/kg, 1300 mg/kg, 1325 mg/kg, 1350 mg/kg, 1375 mg/kg, 1400 mg/kg, 1425 mg/kg, 1450 mg/kg, 1475 mg/kg, 1500 mg/kg, 1525 mg/kg, 1550 mg/kg, 1575 mg/kg, 1600 mg/kg, 1625 mg/kg, 1650 mg/kg, 1675 mg/kg, 1700 mg/kg, 1725 mg/kg, 1750 mg/kg, 1775 mg/kg, 1800 mg/kg, 1825 mg/kg, 1850 mg/kg, 1875 mg/kg, 1900 mg/kg, 1925 mg/kg, 1950 mg/kg, 1975 mg/kg, 2000 mg/kg, 2500 mg/kg, 3000 mg/kg, 3500 mg/kg, 4000 mg/kg, 4500 mg/kg, 5000 mg/kg, 5500 mg/kg, 6000 mg/kg, 6500 mg/kg, 7000 mg/kg, 7500 mg/kg, 8000 mg/kg, 8500 mg/kg, 9000 mg/kg, 9500 mg/kg, 10000 mg/kg, 10500 mg/kg, 11000 mg/kg, 11500 mg/kg, 12000 mg/kg, 12500 mg/kg, 13000 mg/kg, 13500 mg/kg, 14000 mg/kg, 14500 mg/kg, 15000 mg/kg, 15500 mg/kg, 16000 mg/kg, 16500 mg/kg, 17000 mg/kg, 17500 mg/kg, 18000 mg/kg, 18500 mg/kg, 19000 mg/kg, 19500 mg/kg, 20000 mg/kg, or greater.

In another aspect, the dose of a proxidant, salt thereof, or prodrug thereof, comprises 1-100 mg/kg, 1-150 mg/kg, 1-200 mg/kg, 1-250 mg/kg, 1-300 mg/kg, 1-350 mg/kg, 1-400 mg/kg, 1-450 mg/kg, 1-500 mg/kg, 1-550 mg/kg, 1-600 mg/kg, 1-650 mg/kg, 1-700 mg/kg, 1-750 mg/kg, 1-800 mg/kg, 1-850 mg/kg, 1-900 mg/kg, 1-950 mg/kg, 1-1000 mg/kg, 1-1050 mg/kg, 1-1100 mg/kg, 1-1150 mg/kg, 1-1200 mg/kg, 1-1250 mg/kg, 1-1300 mg/kg, 1-1350 mg/kg, 1-1400 mg/kg, 1-1450 mg/kg, 1-1500 mg/kg, 1-1550 mg/kg, 1-1600 mg/kg, 1-1650 mg/kg, 1-1700 mg/kg, 1-1750 mg/kg, 1-1800 mg/kg, 1-1850 mg/kg, 1-1900 mg/kg, 1-1950 mg/kg, 1-2000 mg/kg, 1-2500 mg/kg, 1-3000 mg/kg, 1-3500 mg/kg, 1-4000 mg/kg, 1-4500 mg/kg, 1-5000 mg/kg, 1-5500 mg/kg, 1-6000 mg/kg, 1-6500 mg/kg, 1-7000 mg/kg, 1-7500 mg/kg, 1-8000 mg/kg, 1-8500 mg/kg, 1-9000 mg/kg, 1-9500 mg/kg, 1-10000 mg/kg, 1-10500 mg/kg, 1-11000 mg/kg, 1-11500 mg/kg, 1-12000 mg/kg, 1-12500 mg/kg, 1-13000 mg/kg, 1-13500 mg/kg, 1-14000 mg/kg, 1-14500 mg/kg, 1-15000 mg/kg, 1-15500 mg/kg, 1-16000 mg/kg, 1-16500 mg/kg, 1-17000 mg/kg, 1-17500 mg/kg, 1-18000 mg/kg, 1-18500 mg/kg, 1-19000 mg/kg, 1-19500 mg/kg, 1-20000 mg/kg, 10-100 mg/kg, 10-200 mg/kg, 10-300 mg/kg, 10-400 mg/kg, 10-500 mg/kg, 10-600 mg/kg, 10-700 mg/kg, 10-800 mg/kg, 10-900 mg/kg, 10-1000 mg/kg, 10-1100 mg/kg, 10-1200 mg/kg, 10-1300 mg/kg, 10-1400 mg/kg, 10-1500 mg/kg, 10-1600 mg/kg, 10-1700 mg/kg, 10-1800 mg/kg, 10-1900 mg/kg, 10-2000 mg/kg, 10-2500 mg/kg, 10-3000 mg/kg, 10-3500 mg/kg, 10-4000 mg/kg, 10-4500 mg/kg, 10-5000 mg/kg, 10-5500 mg/kg, 10-6000 mg/kg, 10-6500 mg/kg, 10-7000 mg/kg, 10-7500 mg/kg, 10-8000 mg/kg, 10-8500 mg/kg, 10-9000 mg/kg, 10-9500 mg/kg, 10-10000 mg/kg, 10-10500 mg/kg, 10-11000 mg/kg, 10-11500 mg/kg, 10-12000 mg/kg, 10-12500 mg/kg, 10-13000 mg/kg, 10-13500 mg/kg, 10-14000 mg/kg, 10-14500 mg/kg, 10-15000 mg/kg, 10-15500 mg/kg, 10-16000 mg/kg, 10-16500 mg/kg, 10-17000 mg/kg, 10-17500 mg/kg, 10-18000 mg/kg, 10-18500 mg/kg, 10-19000 mg/kg, 10-19500 mg/kg, 10-20000 mg/kg, including all endpoints, integers and subranges within the disclosed ranges.

### Cancer

The methods described herein can be used with any cancer, for example those described by the National Cancer Institute. The cancer can be a carcinoma, a sarcoma, a myeloma, a leukemia, a lymphoma or a mixed type. Exemplary cancers described by the National Cancer Institute include:
Digestive/gastrointestinal cancers such as anal cancer; bile duct cancer; extrahepatic bile duct cancer; appendix cancer; carcinoid tumor, gastrointestinal cancer; colon cancer; colorectal cancer including childhood colorectal cancer; esophageal cancer including childhood esophageal cancer; gallbladder cancer; gastric (stomach) cancer including childhood gastric (stomach) cancer; hepatocellular (liver) cancer including adult (primary) hepatocellular (liver) cancer and childhood (primary) hepatocellular (liver) cancer; pancreatic cancer including childhood pancreatic cancer; sarcoma, rhabdomyosarcoma; islet cell pancreatic cancer; rectal cancer; and small intestine cancer;
Endocrine cancers such as islet cell carcinoma (endocrine pancreas); adrenocortical carcinoma including childhood adrenocortical carcinoma; gastrointestinal carcinoid tumor; parathyroid cancer; pheochromocytoma; pituitary tumor; thyroid cancer including childhood thyroid cancer; childhood multiple endocrine neoplasia syndrome; and childhood carcinoid tumor;
Eye cancers such as intraocular melanoma; and retinoblastoma;
Musculoskeletal cancers such as Ewing's family of tumors; osteosarcoma/malignant fibrous histiocytoma of the bone; childhood rhabdomyosarcoma; soft tissue sarcoma including adult and childhood soft tissue sarcoma; clear cell sarcoma of tendon sheaths; and uterine sarcoma;
Breast cancer such as breast cancer including childhood and male breast cancer and breast cancer in pregnancy;
Neurologic cancers such as childhood brain stemglioma; brain tumor; childhood cerebellar astrocytoma; childhood cerebral astrocytoma/malignant glioma; childhood ependymoma; childhood medulloblastoma; childhood pineal and supratentorial primitive neuroectodermal tumors; childhood visual pathway and hypothalamic glioma; other childhood brain cancers; adrenocortical carcinoma; central nervous system lymphoma, primary; childhood cerebellar astrocytoma; neuroblastoma; craniopharyngioma; spinal cord tumors; central nervous system atypical teratoid/rhabdoid tumor; central nervous system embryonal tumors; and childhood supratentorial primitive neuroectodermal tumors and pituitary tumor;
Genitourinary cancers such as bladder cancer including childhood bladder cancer; renal cell (kidney) cancer; ovarian cancer including childhood ovarian cancer; ovarian epithelial cancer; ovarian low malignant potential tumor; penile cancer; prostate cancer; renal cell cancer including childhood renal cell cancer; renal pelvis and ureter, transitional cell cancer; testicular cancer; urethral cancer; vaginal cancer; vulvar cancer; cervical cancer; Wilms tumor and other childhood kidney tumors; endometrial cancer; and gestational trophoblastic tumor; Germ cell cancers such as childhood extracranial germ cell tumor; extragonadal germ cell tumor; ovarian germ cell tumor;
Head and neck cancers such as lip and oral cavity cancer; oral cancer including childhood oral cancer; hypopharyngeal cancer; laryngeal cancer including childhood laryngeal cancer; metastatic squamous neck cancer with occult primary; mouth cancer; nasal cavity and paranasal sinus cancer; nasopharyngeal cancer including childhood nasopharyngeal cancer; oropharyngeal cancer; parathyroid cancer; pharyngeal cancer; salivary gland cancer including childhood salivary gland cancer; throat cancer; and thyroid cancer;
Hematologic/blood cell cancers such as a leukemia (e.g., acute lymphoblastic leukemia including adult and childhood acute lymphoblastic leukemia; acute myeloid leukemia including adult and childhood acute myeloid leukemia; chronic lymphocytic leukemia; chronic myelogenous leukemia; and hairy cell leukemia); a lymphoma (e.g., AIDS-related lymphoma; cutaneous T-cell lymphoma; Hodgkin's lymphoma including adult and childhood Hodgkin's lymphoma and Hodgkin's lymphoma during pregnancy; non-Hodgkin's lymphoma including adult and childhood non-Hodgkin's lymphoma and non-Hodgkin's lymphoma during pregnancy; mycosis fungoides; Sezary syndrome; Waldenstrom's macroglobulinemia; and primary central nervous system lymphoma); and other hematologic cancers (e.g., chronic myeloproliferative disorders; multiple myeloma/plasma cell neoplasm; myelodysplastic syndromes; and myelodysplastic/myeloproliferative disorders);
Lung cancer such as non-small cell lung cancer; and small cell lung cancer;
Respiratory cancers such as adult malignant mesothelioma; childhood malignant mesothelioma; malignant thymoma; childhood thymoma; thymic carcinoma; bronchial adenomas/carcinoids including childhood bronchial adenomas/carcinoids; pleuropulmonary blastoma; non-small cell lung cancer; and small cell lung cancer;
Skin cancers such as Kaposi's sarcoma; Merkel cell carcinoma; melanoma; and childhood skin cancer; AIDS-related malignancies;
Other childhood cancers, unusual cancers of childhood and cancers of unknown primary site; and metastases of the aforementioned cancers can also be treated or prevented in accordance with the methods described herein.

The methods described herein may be suited to treat bladder, testicular, ovarian, head and neck, cervical, lung, mesothelioma, esophageal, melanoma, brain tumor, neuroblastoma, colorectal, Wilms' tumor, retinoblastoma, breast, endometrial, adrenocortical, anal, biliary tract, carcinoid tumors, choriocarcinoma, gastric, liver cancer, non-Hodgkin's lymphoma, osteosarcoma, soft-tissue sarcomas, penile, malignant thymoma, anaplastic thyroid cancer, rhabdoid tumor of the kidney, advanced medullary thyroid cancer, carcinoid, mesothelioma, bone, gliomas or prostate cancers. In embodiments, the methods suitably treat bladder cancer (e.g., muscle-invasive bladder carcinoma, advanced or metastatic bladder carcinoma), testicular cancer (e.g., nonseminomatous testicular carcinoma, disseminated seminoma testis or extragonadal germ-cell tumors), ovarian cancer (e.g., ovarian epithelial cancer or ovarian germcell tumors), head and neck cancer (e.g., squamous cell carcinoma), cervical cancer (e.g., invasive, metastatic or recurrent cervical cancer), lung cancer (e.g., small cell lung cancer or non-small cell lung cancer), Wilms' tumor, brain tumors (e.g., gliomas, medulloblastoma or germ cell tumors), neuroblastoma, retinoblastoma, mesothelioma (e.g., malignant pleural mesothelioma), esophageal cancer (e.g., localized or advanced esophageal cancer), and colorectal cancer.

### Modes of Administration

Methods of treatment may include any number of modes of administering a disclosed composition. Modes of administration may include tablets, pills, dragees, hard and soft gel capsules, granules, pellets, aqueous, lipid, oily or other solutions, emulsions such as oil-in-water emulsions, liposomes, aqueous or oily suspensions, syrups, elixirs, solid emulsions, solid dispersions or dispersible powders. For the preparation of pharmaceutical compositions for oral administration, the agent may be admixed with commonly known and used adjuvants and excipients such as for example, gum arabic, talcum, starch, sugars (such as, e.g., mannitose, methyl cellulose, lactose), gelatin, surface-active agents, magnesium stearate, aqueous or non-aqueous solvents, paraffin derivatives, cross-linking agents, dispersants, emulsifiers, lubricants, conserving agents, flavoring agents (e.g., ethereal oils), solubility enhancers (e.g., benzyl benzoate or benzyl alcohol) or bioavailability enhancers (e.g., Gelucire™). In the pharmaceutical composition, the agent may also be dispersed in a microparticle, e.g., a nanoparticulate composition.

For parenteral administration, the agent can be dissolved or suspended in a physiologically acceptable diluent, such as, e.g., water, buffer, oils with or without solubilizers, surface-active agents, dispersants or emulsifiers. As oils for example and without limitation, olive oil, peanut oil, cottonseed oil, soybean oil, castor oil and sesame oil may be used. More generally spoken, for parenteral administration, the agent can be in the form of an aqueous, lipid, oily or other kind of solution or suspension or even administered in the form of liposomes or nano-suspensions.

The term “parenterally,” as used herein, refers to modes of administration which include intravenous, intramuscular, intraperitoneal, intrasternal, subcutaneous and intraarticular injection and infusion.

Various embodiments and aspects of the inventions described herein are summarized by the following clauses:

Clause 1. A combination therapeutic comprising:
a therapeutically effective amount of one or more antineoplastic agents, or pharmaceutically acceptable salts or esters thereof, and
a therapeutically effective amount of one or more pro-oxidants, or pharmaceutically acceptable salts or esters thereof.

Clause 2. The combination therapeutic of clause 1, wherein the one or more antineoplastic agents is an alkylating agent or a pro-drug of an alkylating agent.

Clause 3. The combination therapeutic of clause 1 or 2, wherein the one or more antineoplastic agents has a structure:

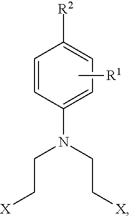

wherein
X, at each occurrence, is independently halo;
R¹, at each occurrence, is independently hydrogen, C₁₋₆alkyl, C₁₋₆alkylene, C₁₋₆haloalkyl, cyano, —OR^{1a}, —SR^{1a}, —CO₂R^{1a}, —C(O)R^{1a}, —SO₂R^{1b}, —N(R^{1b})₂, —CO₂N(R^{1b})₂, or —NO₂;
R² is BH(OR^{1b}), B(OR^{1b})₂,

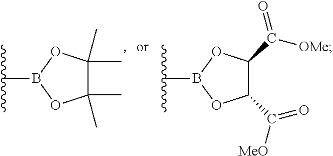

R^{1a}, at each occurrence, is independently hydrogen, C₁₋₆alkyl, or C₁₋₂haloalkyl; and
R^{1b}, at each occurrence, is independently hydrogen or C₁₋₆alkyl.

Clause 4. The combination therapeutic of any one of clauses 1-3, wherein the one or more antineoplastic agents is FAN-NM-CH₃.

Clause 5. The combination therapeutic of any one of clauses 1-4, wherein the one or more antineoplastic agents is a DNA crosslinking agent or a pro-drug of a DNA crosslinking agent.

Clause 6. The combination therapeutic of any one of clauses 1-5, wherein the one or more antineoplastic agent is selected from the group comprising:

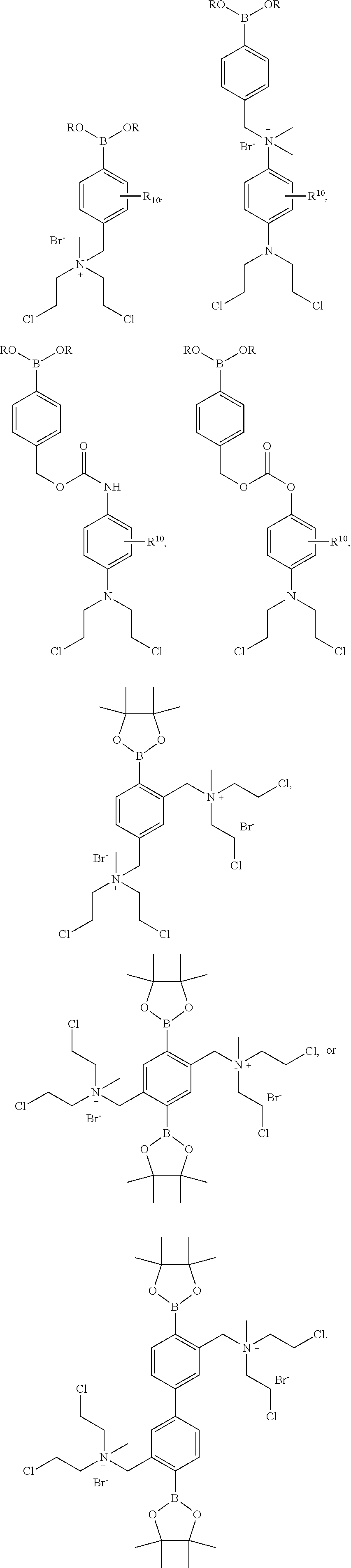

wherein R¹⁰, at each occurrence, is independently hydrogen, C₁₋₆alkyl, C₁₋₆alkylene, C₁₋₆haloalkyl, cyano, —OR^{1a}, —SR^{1a}, —CO₂R^{1a}, —C(O)R^{1a}, —SO₂R^{1b}, —N(R^{1b})₂, —CO₂N(R^{1b})₂, —NO₂, or —N(RR^{1b})—OR^{1a};
R^{1a}, at each occurrence, is independently hydrogen, C₁₋₆alkyl, or C₁₋₂haloalkyl; and
R^{1b}, at each occurrence, is independently hydrogen or C₁₋₆alkyl.

Clause 7. The combination therapeutic of any one of clauses 1-6, wherein the one or more antineoplastic agents comprise one or more chemotherapeutic agents.

Clause 8. The combination therapeutic of any one of clauses 1-7, wherein the one or more chemotherapeutic agents comprise adriamycin, anthracyclines, bleomycin, or cisplatin, their H₂O₂-activated prodrugs, or combinations thereof.

Clause 9. The combination therapeutic of any one of clauses 1-8, wherein the one or more pro-oxidants is a reactive oxygen species amplifying agent.

Clause 10. The combination therapeutic of any one of clauses 1-9, wherein the one or more pro-oxidants comprise compounds containing quinone moieties.

Clause 11. The combination therapeutic of any one of clauses 1-10, wherein the one or more pro-oxidants comprise vitamin C, polyphenol, hydrogen peroxide, carotenoids (Lutein, β-carotene, Astaxanthin, Fucoxanthin, β-Cryptoxanthin, Bixin, and lycopene), diallyl trisulfide (DATS), indomethacin (indo), Piperlongumine, Vitamine E, β-Lapachone, Plumbagin, Arsenic Trioxide, Quercetin, Cinnamaldehyde, Bisdemethoxycurcumin (Curcumin), (−)-Epigallocatechin gallate, Gensenosides (Rg3, Rh2), 2-methoxyestradiol, Emodin, β-phenylethyl isothiocyanate, nonsteroidal anti-inflammatory drugs (NSAIDs), or combinations thereof.

Clause 12. A method for treating a disease or disorder, the method comprising:
sequentially or simultaneously administering to a subject in need thereof
a therapeutically effective amount of one or more antineoplastic agents, or pharmaceutically acceptable salts or esters thereof, and
a therapeutically effective amount of one or more pro-oxidants, or pharmaceutically acceptable salts or esters thereof; and
repeating the administration until the disease or disorder is treated, ameliorated, or symptoms are reduced.

Clause 13. The method of clause 12, wherein the disease or disorder is a cancer.

Clause 14. The method of clauses 12 or 13, wherein the disease or disorder is a solid cancer.

Clause 15. The method of any one of clauses 12-14, wherein the cancer is a breast cancer, a glioblastoma, leukemia, lung cancer, or renal cancer.

Clause 16. The method of any one of clauses 12-15, wherein the disease or disorder is a cancer associated with oxidative stress.

Clause 17. The method of any one of clauses 12-16, wherein the disease or disorder is an inflammatory disease.

Clause 18. The method of any one of clauses 12-17, wherein the inflammatory disease is arthritis.

Clause 19. The method of any one of clauses 12-18, wherein administering comprises intraperitoneal injection, intramuscular injection, subcutaneous injection, intravenous injection, intrathecal infusion, oral administration, or a combination thereof.

Clause 20. The method of any one of clauses 12-19, wherein the one or more pro-oxidants increase an amount of reactive oxygen species in a cancerous cell.

Clause 21. The method of any one of clauses 12-20, wherein the one or more antineoplastic agents is active in the presence of reactive oxygen species.

Clause 22. The method of any one of clauses 12-21, wherein a therapeutically effective amount of a one or more pro-oxidants and one or more antineoplastic agents reduces a malignant neoplasm size, volume, mass, or a combination thereof.

Clause 23. The method of any one of clauses 12-22, wherein the one or more pro-oxidants is added at a period of time prior to addition of the antineoplastic agent.

Clause 24. The method of any one of clauses 12-23, wherein the period of time is 1-3 hours.

Clause 25. Use of one or more antineoplastic agents and one or more pro-oxidants, or pharmaceutically acceptable salts or esters thereof, as a medicament for the treatment of cancer or an inflammatory disease in subject in need thereof.

Clause 26. A kit comprising:
one or more antineoplastic agents, or pharmaceutically acceptable salts or esters thereof;
one or more pro-oxidants, or pharmaceutically acceptable salts or esters thereof;
optionally a device or means for administering the antineoplastic agent and pro-oxidant;
optionally tamper resistant packaging; and
optionally, a label or instructions for use thereof.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

### EXAMPLES

### Antineoplastic Agents

FAN-NM-CH₃ and derivatives thereof and their synthesis are disclosed in U.S. Pat. No. 8,962,670, which is incorporated by reference herein for such teachings.

### Cell Culture

The human tumor cell line MDA-MB-468 (HTB-132), MCF7 (HTB-22), MDA-MB-436 (HTB-130), MDA-MB-231 (HTB-26) and normal cell lines HMEC (PCS-600-010), MCF 10A (CRL-10317) were purchased from the American Type Culture Collection. U-87 MG cells were generously provided by Dr. Shama Mirza (Shimadzu Laboratory). MDA-MB-468, MDA-MB-436, and MDA-MB-231 cells were cultured in L-15 Leibovitz media (Thermo Scientific Catalog: 41300070) supplemented with 10% fetal bovine serum (FBS, Biowest: S1620), 1% non-essential amino acids (NEAA 100× solution, HyClone no: SH30238.01), and 1% penicillin and streptomycin (HyClone Penicillin Streptomycin 100× Solution, HyClone no: SV30010) at 37° C. in 100% relative humidity. MCF7 and U-87 MG cells were maintained in ATCC-formulated Eagle's Minimum Essential Medium (30-2003) supplemented with 10% fetal bovine serum (FBS, Biowest: S1620). One percent penicillin and streptomycin (HyClone Penicillin Streptomycin 100× Solution, HyClone no: SV30010) and 0.01 mg/ml human recombinant insulin (Sigma Aldrich Inc: 91077C) were added to MCF7 media. HMEC cells were maintained in Mammary Epithelial Cell Growth Media Kit (PCS-600-030, PCS-600-040) from ATCC. MCF 10A cells were maintained in Lonza media kit MEGM (CC-3150) supplemented with 100 ng/ml Cholera toxin. MCF7, U-87 MG, HMEC, and MCF 10A cells were kept in 5% CO₂ incubator at 37° C.

### Cytotoxicity Assays

### FAN-NM-CH₃ Dose Response

Cells were plated into 384-well optical bottom plates (Nunc: 142762) in 40 μL at densities ranging from 5,000 to 10,000 cells/well. The plates were incubated for 3 h prior to the addition of the compounds. FAN-NM-CH₃ was solubilized in dimethyl sulfoxide (DMSO) at 20 mM stock and serially diluted (2-fold). 400 nL of the serially diluted stocks were added to the cell plate (1:100 dilution) using a Tecan Freedom EVO liquid handling system equipped with a 100 nL pin tool (V&P Scientific). Plates were incubated for an additional 48 h followed by the addition of 40 μL of Celltiter-Glo Reagent (Promega). Luminescence was measured after 30 minutes of incubation using an Infinite M1000 (Tecan) plate reader.

### Pro-Oxidant Dose Response

Cells were plated into 384-well optical bottom plates (Nunc: 142762) in 40 μL (Final reaction volume) at densities ranging from 5,000 to 10,000 cells/well. The plates were incubated for 3 h prior to the addition of the compound. Epigallocatechin Gallate (989-51-5), Piperlogumine (20069-09-4), β-lapachone (4707-32-8), Quercetin (117-39-5), Propyl Gallate (121-79-9), and Vitamin E (10191-41-0) were dissolved in DMSO at 20 mM stock. Arsenic (III) Oxide (1327-53-3) and Vitamin C (BDH9242) were dissolved in Millipore water. Arsenic stock was 20 mM and Vitamin C was dissolved at 1 M and pH was adjusted to 7 pH with 1 M Sodium Hydroxide solution. All the above stock solutions were serially diluted (2-fold). 400 nL of serially diluted prooxidants were added to the cell plate. Plates were incubated for an additional 48 h followed by the addition of 40 L of Celltiter-Glo Reagent (Promega). Luminescence was measured after 30 minutes of incubation using an Infinite M1000 (Tecan) plate reader.

Combination Effect: FAN-NM-CH₃ Dose Response—with Specific Ascorbic Acid Dose

Cells were plated into 384-well optical bottom plates (Nunc: 142762) in 40 μL (Final reaction volume) at densities ranging from 5,000 to 10,000 cells/well. The plates were incubated for 3 h prior to the addition of the compounds. Ascorbic Acid (VWR: BDH9242-100G) was dissolved in Millipore water at 0.5 M and pH was adjusted to pH 7 with 1 M sodium hydroxide solution. This stock was further diluted to have final safe dose stocks (500 mM, 400 mM, 120 mM, and 100 mM). 100 nL of these diluted ascorbic acid stocks were added to the cell plate (1:400 dilution). The plates were incubated for 1 h prior to the addition of FAN-NM-CH₃. FAN-NM-CH₃ was solubilized in dimethyl sulfoxide (DMSO) at 20 mM stock and serially diluted (2-fold). 400 nL of the serially diluted stocks were added to the cell plate (1:100 dilution) using a Tecan Freedom EVO liquid handling system equipped with a 100 nL pin tool (V&P Scientific). Plates were incubated for an additional 48 h followed by the addition of 40 μL of Celltiter-Glo Reagent (Promega). Luminescence was measured after 30 minutes of incubation using an Infinite M 1000 (Tecan) plate reader.

Combination Effect with FAN-NM-CH₃, Ascorbic Acid, and Catalase

Cells were plated into 384-well optical bottom plates (Nunc™ Catalog: 142762) in 40 μL (Final reaction volume) at densities ranging from 5,000 to 10,000 cells/well. The plates were incubated for 3 h prior to the addition of the catalase. 100 nL of 7 μM Catalase solution (Sigma-Aldrich: C3155-100 MG) was added to the cell plate (1:400). The plates were incubated for 1 h prior to the addition of ascorbic acid. Ascorbic Acid (VWR: BDH9242-100G) was dissolved in Millipore water at 0.5 M and pH was adjusted to pH 7 with 1 M sodium hydroxide solution. This solution was diluted to 400 mM, 200 mM, 100 mM, and 25 mM stocks. 400 nL of the diluted ascorbic acid stocks were added to the cell plate (1:100 dilution). Plates were incubated for 1 h prior to the addition of the FAN-NM-CH₃. FAN-NM-CH₃ was solubilized in dimethyl sulfoxide (DMSO) at 2 mM, 1.2 mM, 0.8 mM, 0.4 mM, and 0.2 mM stocks. 100 nL of this stock was added to the cell plate (1:400 dilution) using a Tecan Freedom EVO liquid handling system equipped with a 100 nL pin tool (V&P Scientific). Plates were incubated for an additional 48 h followed by the addition of 40 μL of Celltiter-Glo Reagent (Promega). Luminescence was measured after 30 minutes of incubation using an Infinite M1000 (Tecan) plate reader.

### Measurement of Catalase Activity

Catalase activity was measured with a commercially available Catalase Colorimetric Activity Kit (Invitrogen: EIACATC). This Assay was performed according to manufacturer's protocol. Cells were cultured in a clear 6-well plate. 1×10⁶ cells were harvested using a rubber policeman in 1 mL of cold PBS buffer. Cells were centrifuged in suspension at 250×g for 10 minutes at 4° C. The supernatant was discarded and the cell pellet was centrifuged in 1 mL of cold 1× assay buffer at 10,000×g for 15 minutes at 4° C. Supernatant was collected and used immediately. 25 μL of catalase standards and collected samples were added to the clear 96-well Half Area plate provided with the kit. 25 μL of hydrogen peroxide reagent was added to each well and plate was incubated at room temperature for 30 minutes. 25 μL of substrate was added to each well and followed by 25 μL of 1×HRP solution into each well. The plate was then incubated at room temperature for 15 minutes. Absorbance was measured at 560 nm using an Infinite M1000 (Tecan) plate reader.

Extracellular H₂O₂ Measurement

The extracellular H₂O₂ levels were determined using an Amplex Red Hydrogen Peroxide Assay (Invitrogen, A22188) as per the manufacturer's protocol.

Ascorbic Acid Dose Dependent H₂O₂ Release

25×10³-50×10³ cells were plated into 384 Well Black, Optically Clear Polymer Bottom Plate (Thermo Scientific Catalog: 142761) in 40 μL (Final reaction volume). The plates were incubated for 3 h prior to the addition of the compound. Ascorbic Acid (VWR: BDH9242-100G) was dissolved in Millipore water at 0.5 M and pH was adjusted to pH 7 with 1 M sodium hydroxide solution. This solution was serially diluted (2-fold). 800 nL of the serially diluted ascorbic acid was added to the cell plate (1:50 dilution). Plates were incubated for an additional 48 h.

H₂O₂ Levels in Combination Treatments

25×10³-50×10³ cells were plated into 384 Well Black, Optically Clear Polymer Bottom Plate (Thermo Scientific Catalog: 142761) in 40 μL (Final reaction volume). The plates were incubated for 3 h prior to the addition of the catalase. 100 nL of 7 μM Catalase solution (Sigma-Aldrich: C3155-100 MG) was added to the cell plate (1:400). The plates were incubated for 1 h prior to the addition of ascorbic acid. Ascorbic Acid (VWR: BDH9242-100G) was dissolved in Millipore water at 0.5 M and pH was adjusted to pH 7 with 1 M sodium hydroxide solution. This solution was diluted to 400 mM, 200 mM, 100 mM, and 25 mM stocks. 400 nL of the diluted ascorbic acid stocks were added to the cell plate (1:100 dilution). Plates were incubated for 1 h prior to the addition of the FAN-NM-CH₃. FAN-NM-CH₃ was solubilized in dimethyl sulfoxide (DMSO) at 2 mM, 1.2 mM, 0.8 mM, 0.4 mM, and 0.2 mM stocks. 100 nL of this stock was added to the cell plate (1:400 dilution) using a Tecan Freedom EVO liquid handling system equipped with a 100 nL pin tool (V&P Scientific). Plates were incubated for an additional 48 h.

After 48 h of incubation, the cells were washed twice with 1×KRPG buffer (Krebs-Ringer phosphate consists of 145 mM NaCl, 5.7 mM sodium phosphate, 4.86 mM KCl, 0.54 mM CaCl₂, 1.22 mM MgSO₄, 5.5 mM glucose, pH 7.35) and incubated for 5 h in 40 μL KRPG buffer. Then, 20 μL of this KRPG buffer was transferred into another 384 Well Black, Optically Clear Polymer Bottom Plate (Thermo Scientific™ Catalog: 142761) in triplicate and mixed with an equal amount of Amplex Red reagent (50 μM Amplex Red and 0.1 U/mL HRP final concentrations). After 5 h incubation, fluorescence was measured (Ex/Em: 560/590 nm) on an infinite M1000 (Tecan) microplate reader.

The final concentration of H₂O₂ was adjusted to two-fold to account for the dilution by the same volume of amplex red reagent.

Total fluorescence intensity divided by the number of cells alive was calculated to report fluorescence intensity per cell. Viability was measured using Promega CellTiter Glow reagent. Intracellular H₂O₂ Measurements

H₂O₂ levels were determined by a Hydrogen Peroxide Assay kit from Abcam (ab138874). The assay was performed as per the manufacturer's protocol. The kit uses AbGreen indicator that is cell-permeable and produces green fluorescence when reacts with hydrogen peroxide in live cells.

### Ascorbic Acid Dose Dependent ROS Release

25×10³-50×10³ cells were plated into 384 Well Black, Optically Clear Polymer Bottom Plate (Thermo Scientific Catalog: 142761) in 40 μL (Final reaction volume). The plates were incubated for 3 h prior to the addition of the compound. Ascorbic Acid (VWR: BDH9242-100G) was dissolved in Millipore water at 0.5 M and pH was adjusted to pH 7 with 1 M sodium hydroxide solution. This solution was serially diluted (2-fold). 800 nL of the serially diluted ascorbic acid was added to the cell plate (1:50 dilution). Plates were incubated for an additional 48 h.

### ROS Levels in Combination Treatment

25×10³-50×10³ cells were plated into 384 Well Black, Optically Clear Polymer Bottom Plate (Thermo Scientific Catalog: 142761) in 40 μL (Final reaction volume). The plates were incubated for 3 h prior to the addition of the catalase. 100 nL of 7 μM Catalase solution (Sigma-Aldrich: C3155-100 MG) was added to the cell plate (1:400). The plates were incubated for 1 h prior to the addition of ascorbic acid. Ascorbic Acid (VWR: BDH9242-100G) was dissolved in Millipore water at 0.5 M and pH was adjusted to pH 7 with 1 M sodium hydroxide solution. This solution was diluted to 400 mM, 200 mM, 100 mM, and 25 mM stocks. 400 nL of the diluted ascorbic acid stocks were added to the cell plate (1:100 dilution). Plates were incubated for 1 h prior to the addition of the FAN-NM-CH₃. FAN-NM-CH₃ was solubilized in dimethyl sulfoxide (DMSO) at 2 mM, 1.2 mM, 0.8 mM, 0.4 mM, and 0.2 mM stocks. 100 nL of this stock was added to the cell plate (1:400 dilution) using a Tecan Freedom EVO liquid handling system equipped with a 100 nL pin tool (V&P Scientific). Plates were incubated for an additional 48 h.

After 48 h of incubation, the cells were washed twice with PBS buffer. 25 μL of 1× AbGreen indicator working solution from Abcam (ab138874) was added to each well and incubated for 30 minutes at 37° C. in the dark. Fluorescence was measured (Ex/Em: 490/520 nm) on an infinite M1000 (Tecan) microplate reader. Fluorescence microscope Images were also captured using GFP Light cube on EVOS FL Digital Inverted Microscope.

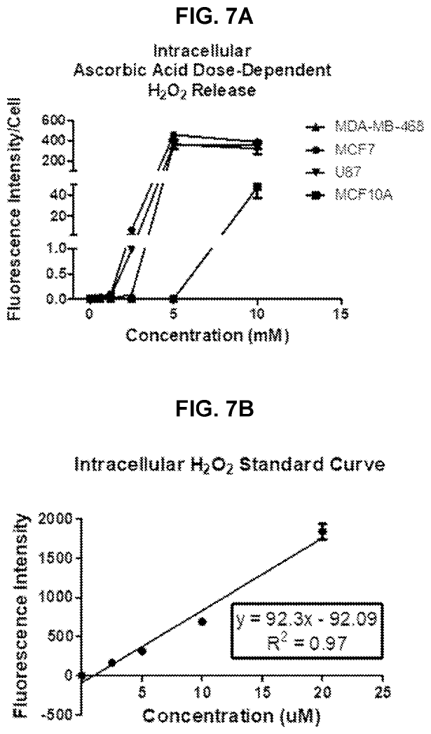

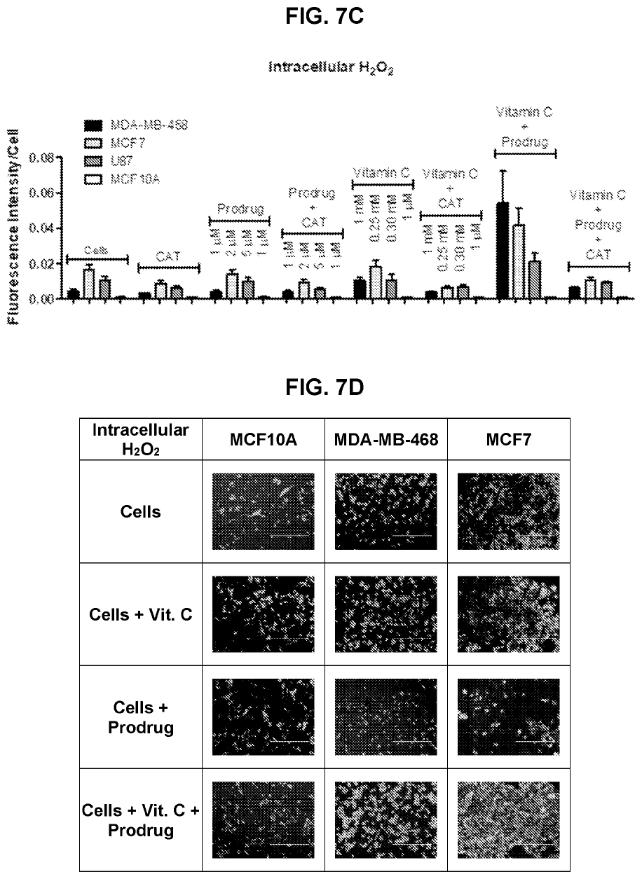

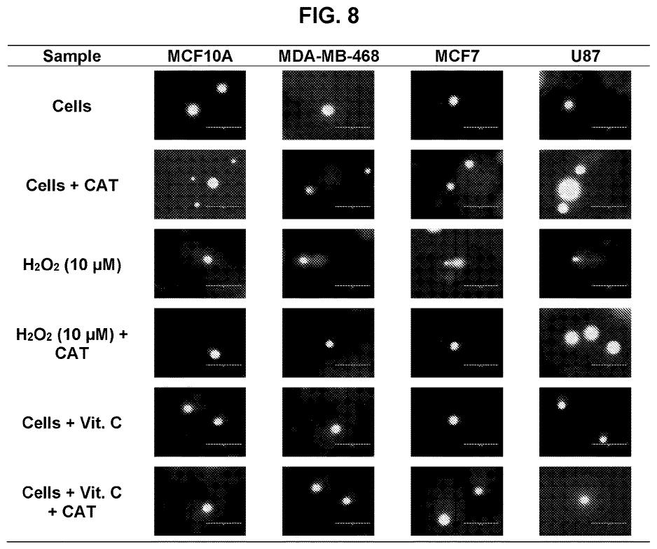

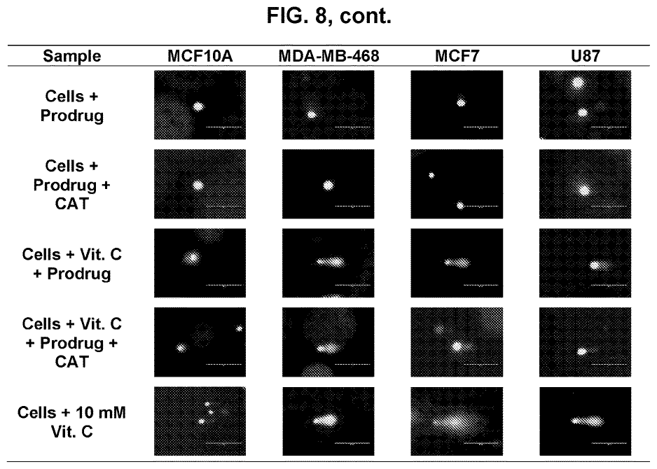

Data in FIGS. 7A and 7C was adjusted using the equation, Total fluorescence intensity/number of cells alive to report fluorescence intensity per cell. Viability was measured using Promega CellTiter Glow reagent.

### Alkaline Comet Assays

The comet assay was performed according to manufacturer's protocol (Abcam: ab238544) with minor adjustments. Cells were seeded in 6-well Tissue culture, surface treated, sterile, clear plates (VWR: 10062-892) at a cell density of 10⁵ cells per well. Upon 90% confluency, cells were treated at varied conditions for 48 h. Cells were gently removed from the 6-well plate by scraping with a rubber policeman in 1 mL ice-cold PBS (without Mg²⁺ and Ca²⁺). Cell suspension was centrifuged at 700×g for 5 mins. Supernatant was discarded. Finally, cells were resuspended in PBS and further diluted to obtain 1×10⁵ cells/mL. Cell samples (20 μL) were mixed gently with the warm agarose (180 L, 37° C.). 150 μL/well of this mix was transferred onto a pre-warmed glass slide and maintained at 4° C. in the dark for 30 minutes to let the agarose solidify. The slides were carefully immersed into a small basin containing pre-chilled lysis buffer at 4° C. for 2 h in the dark to lyse the cell membrane. The Lysis buffer was then replaced with pre-chilled alkaline unwinding solution (300 mM NaOH, 1 mM EDTA) at 4° C. for 30 minutes in the dark to denature DNA. The slides were then gently transferred into a horizontal electrophoresis chamber filled with pre-chilled alkaline electrophoresis solution (300 mM NaOH, 1 mM EDTA, pH>13). A voltage of 35 V was applied for 30 minutes. The slides were then removed and immersed slowly to rinse twice with pre-chilled DI water for 2 minutes followed by cold 70% ethanol for 5 minutes. The slides were allowed to air dry for 1 h in the dark. 100 μL/well of diluted Vista Green DNA dye was added onto the agarose. The slides were then incubated at room temperature for 15 minutes. Comets were analyzed under an EVOS FL Digital Inverted Microscope at 20× magnification and DNA damage was qualified using TriTek CometScore Software.

### Animals

Six-week-old female CD1 mice (Charles River Laboratory) were used for a safety study. Immune-deficient female nude mice (Charles River Strain, Code 490) weighing 22-25 g were used for an in vivo efficacy study. The animals were housed under specific pathogen-free conditions, under standard conditions of humidity, temperature, and a controlled 12 h light and dark cycle, and had free access to food and water. All animals were allowed a period of adaptation (~7 d) before experimental procedures. All animal experiments were in compliance with the University of Wisconsin-Milwaukee Institutional Animal Care and Use Committees (IACUC).

### Safety Studies

The maximum tolerated dose (MTD), defined as the highest dose not causing a serious adverse event (e.g., death, convulsion, ataxia, aberrant behavior, or evident pain) observed within 2 d of observation, was determined for the prodrug and Vitamin C among female CD1 mice using groups of three animals per group. Vitamin C was dissolved in DI water and pH was adjusted to 7. 100 μL of this was administered intraperitonially. The prodrug was formulated in a mixture of DMSO, poly(ethylene glycol) (PEG) 400, and phosphate-buffered saline (PBS) (volume ratio 2:19:19). 1 h after the Vitamin C injection, 100 μL of the prodrug was administered through IP. 3 mice per group were used with escalating IP dosages of the Vitamin C (1 g/kg, 2 g/kg, 3 g/kg, and 4 g/kg) against fixed prodrug doses (5 mg/kg, 10 mg/Kg, and 20 mg/kg) until serious adverse events were observed or the maximum dosage was reached (20 mg/kg prodrug in combination with 4 g/kg Vitamin C). Dose escalations were conducted with a one-day interval, and weights were documented on the second day. Once the dosing was completed, animals were observed for another 2 d to observe delayed-onset toxicity effects. Animals with the following signs were euthanized: weight loss of 20% from the initial weight or more, the inability to rise, ambulate, or reach food and water for over 3 d, and the presence of a labored respiration. To identify a safe dose of the Vitamin C for an in vivo efficacy study, decreased doses of Vitamin C (500 mg/kg, 750 mg/kg, and 1 g/kg) and the prodrug (5 mg/kg, 10 mg/kg, and 20 mg/kg) (IP injection) were given to the female CD-1 mouse (three mice for each dose) each day until a dose was administered with no signs of weight loss for all mice over a period of 5 days.

### In Vivo Xenograft Efficacy Study

Seven-week-old Immune-deficient female nude mice were anesthetized with isoflurane and injected subcutaneously with cancer cells (MDA-MB-468) suspended in a 1:1 solution of matrigel and Dulbecco's Modified Eagle Medium (DMEM) media. All cancer cells were obtained from the American Type Culture Collection (ATCC) and were negative for bloodborne pathogens. Cell numbers for each inoculation (100 μL per mouse to the subcutaneous area of the flank) were 5×10⁶. Animals were monitored daily for palpable tumors, and animal weights were recorded weekly before the compound was administered. When the tumors reached treatment size (200 mm³), the mice were randomized to treatment groups (4 groups with 3 mice per group). A vehicle group, Vitamin C group, prodrug group, and the combination group. Each was given IP doses each day (5 d per week) for seven weeks. For the combination group, Vitamin C IP injection was given 1 h prior to the prodrug IP injection. Vitamin C was dissolved in DI water and pH adjusted to 7. The prodrug was formulated in a mixture of DMSO, poly(ethylene glycol) (PEG) 400, and phosphate-buffered saline (PBS) (volume ratio 2:19:19). The volume of injection for both compounds was 100 μL at a concentration of 3.0 mg/kg of the prodrug and 500 mg/kg of the Vitamin C. Mice were regularly weighed, and tumor sizes were measured using electronic calipers every 7 d. After the tumors shrink to <25 mm³, all tumors were harvested, weighed, and stored in −80° C. Table 1 shows the potential dosages of pro-oxidant and prodrugs.

TABLE 1
Possible Routes of Administration and Dose Ranges in Mice

| | Pro-oxidant | Prodrug |
|---|---|---|
| Intratumoral Injection/ | 1-500 mg/kg | 0.1-20 mg/kg |
| Localized injection | | |
| Intravenous (IV) | 2.5-500 mg/kg | 0.1-20 mg/kg |
| Injection | | |
| Intraperitoneal (IP) | 10-10000 mg/kg | 1.0-20 mg/kg |
| Injection | | |

### FAN-NM-CH₃ Alone Demonstrates Minimal Toxicity Towards Normal Cells and Significant Toxicity Towards Cancer Cells

Table 2 shows half-maximal inhibitory concentration (IC₅₀) of FAN-NM-CH₃ for various cell types. The IC₅₀ values align with previously reported findings. FAN-NM-CH₃ exhibited greater toxicity towards diverse tumor cell lines while demonstrating lower toxicity towards normal cells. Notably, MDA-MB-468 exhibited significantly higher cytotoxicity, with an IC₅₀ value of 3.02 μM. This value is 2-fold lower than the IC₅₀ value observed for MCF7 cells (6.6 μM). Conversely, FAN-NM-CH₃ displayed lowered toxicity towards U-87 MG cells, with an IC₅₀ value of 24.8 μM. Normal cells (i.e., MCF 10A) exhibited the least toxicity in response to FAN-NM-CH₃, with an IC₅₀ of 48 μM. The prodrug's properties suggests that the increased cytotoxicity observed in tumor cells could be attributed to heightened oxidative stress in comparison to normal cells, which experience lower oxidative stress levels.

TABLE 2
FAN-NM-CH₃ Cytotoxicity

| | Prodrug | Prodrug + Vitamin | Vitamin C (MTD) |
|---|---|---|---|
| Cell Line | IC₅₀ (μM) | C IC₅₀ (μM) | (mM) |
| MDA-MB-486 | 3.02 ± 0.2 | 0.49 ± 0.05 | 1 |
| MCF7 | 6.60 ± 0.5 | 2.14 ± 0.3 | 0.25 |
| U87 | 24.83 ± 1.5 | 12.73 ± 0.8 | 0.3 |
| MCF10A (N) | 48.01 ± 3.5 | 35.87 ± 2.6 | 1.25 |
| MDA-MB-468 + ChL | 30.17 ± 2.3 | 28.66 ± 2.4 | 1 |

### Pro-Oxidant Enhancement of FAN-NM-CH₃

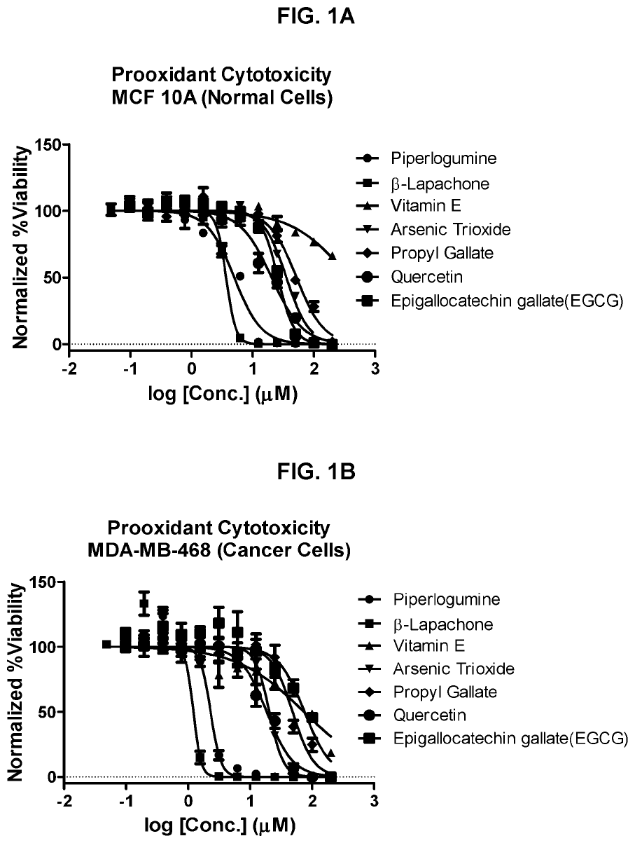

The cytotoxicity of a series of pro-oxidants (Table 3) were tested as a single agent or in combination with FAN-NM-CH₃. The dose dependent cytotoxicity of the pro-oxidants was determined in MCF 10A (normal cells) and MDA-MB-468 (cancer cells). The dose-response curves are shown in FIGS. 1A and 1B.

TABLE 3
Exemplary Pro-oxidants

| Prooxidant | Structure |
|---|---|
| Vitamin C | 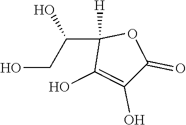 [SMILES: O=C1O[C@H]([C@@H](O)CO)C(O)=C1O] |
| Piperlogumine | 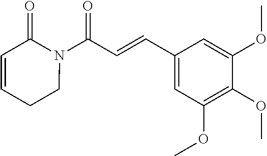 [SMILES: COc1cc(/C=C/C(=O)N2CCC=CC2=O)cc(OC)c1OC] |
| B-Lapachone | 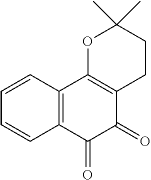 [SMILES: CC1(C)CCC2=C(O1)c1ccccc1C(=O)C2=O] |
| Vitamin E | 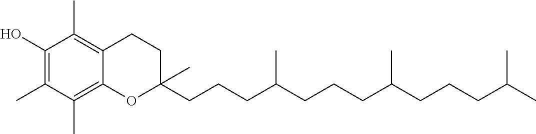 [SMILES: Cc1c(C)c2c(c(C)c1O)CCC(C)(CCCC(C)CCCC(C)CCCC(C)C)O2] |
| Arsenic Trioxide | 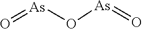 |
| Propyl Gallate | 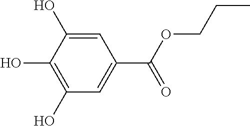 |
| Quercetin | 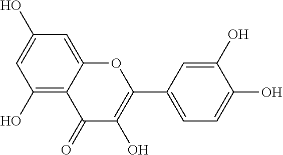 [SMILES: O=c1c(O)c(-c2ccc(O)c(O)c2)oc2cc(O)cc(O)c12] |
| EGCG | 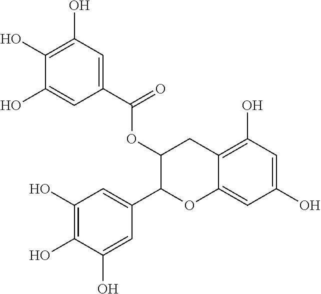 [SMILES: O=C(OC1Cc2c(O)cc(O)cc2OC1c1cc(O)c(O)c(O)c1)c1cc(O)c(O)c(O)c1] |

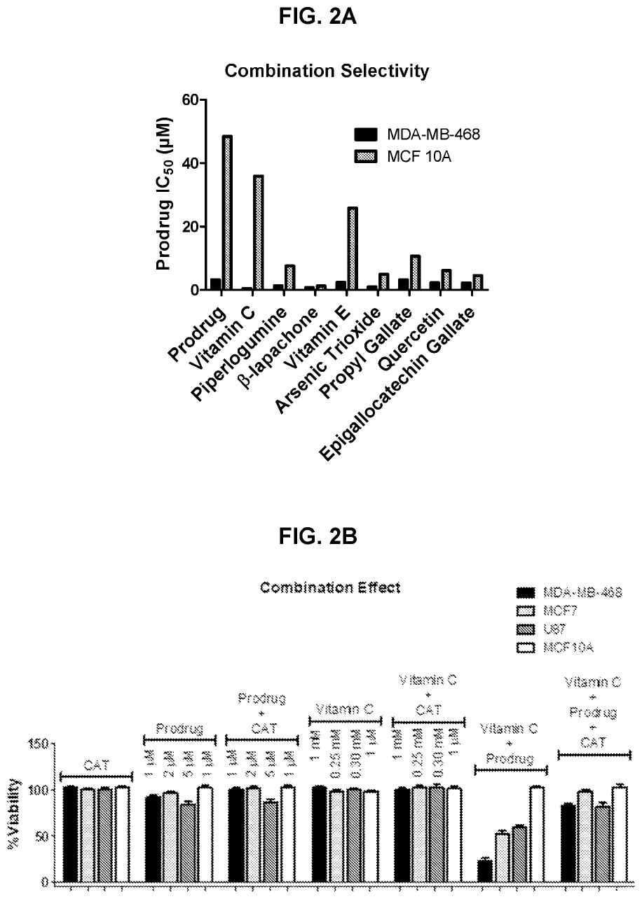

Most pro-oxidants enhanced the cytotoxicity of FAN-NM-CH₃ with decreased IC₅₀ in most cancer cell lines. Among different prooxidants, vitamin C showed the highest synergistic anticancer effect in combination with FAN-NM-CH₃ and the lowest cytotoxicity towards normal cells, thus leading to the highest selectivity (SI) towards cancer cells. The SI (Selectivity Index, the ratio of IC₅₀ value in Normal MCF 10A cells to that in MDA-MB-468 cancer cells) is 2 for vitamin C alone, 15 for prodrug FAN-NM-CH₃ alone, and 73 for combination of vitamin C and FAN-NM-CH₃. Combination of other prooxidants with FAN-NM-CH₃ showed better selectivity than pro-oxidants alone but lower selectivity than FAN-NM-CH₃ alone (Table 4 and Table 5). A comparison between the IC₅₀ values of different pro-oxidants and FAN-NM-CH₃ in normal and cancerous cells can be used to show the selectivity towards cancer cells when used as a monotherapy (FIG. 2).

TABLE 4
IC₅₀ (μM) of Pro-oxidants in Cancer and Normal Cells

| | Cancer Cells | | Normal Cells | | |
|---|---|---|---|---|---|
| | | MSD^{b} | | MSD^{c} | |
| | Mean ± SD | (μM) | Mean ± SD | (μM) | SI*^{a} |
| Prodrug | 3.02 ± 0.2 | 1 | 48.40 ± 3.1 | 6 | 15 |
| Vitamin C | 2.33 ± 0.3 | 1 mM | 4.20 ± 0.4 | 2 mM | 2 |
| Piperlo- | 2.24 ± 0.05 | 0.78 | 4.91 ± 0.51 | 1.56 | 2 |
| gumine | | | | | |
| β-Lapachone | 1.06 ± 0.17 | 0.78 | 3.42 ± 0.22 | 1.56 | 3 |
| Vitamin E | 76.5 ± 6.62 | 1.56 | 375.82 ± 21.4 | 25.00 | 5 |
| Arsenic | 6.27 ± 0.33 | 0.79 | 8.52 ± 0.40 | 1.56 | 1 |
| Trioxide | | | | | |
| Propyl | 49.23 ± 1.83 | 25.00 | 51.35 ± 5.45 | 12.50 | 1 |
| Gallate | | | | | |
| Quercetin | 15.83 ± 2.15 | 3.13 | 19.59 ± 1.24 | 6.25 | 1 |
| EGCG | 64.97 ± 3.45 | 12.50 | 28.51 ± 2.55 | 6.25 | 0.5 |

^{a}SI: Selectivity Index is defined as the ratio of IC50 value in Normal MCF 10A cells to that in MDA-MB-468 cancer cells.
^{b}The Maximum Safe Dose (MSD) refers to the highest dose of the prooxidant identified as safe, exhibiting no discernible toxicity towards the tested cells.

TABLE 5
IC₅₀ (μM) of Prodrug Combined with
Maximum Safety Dose of Pro-oxidant

| | Cancer Cells | Normal Cells | SI*^{b} |
|---|---|---|---|
| Vitamin C | 0.49 | 35.9 | 73 |
| Piperlogumine | 1.31 | 7.58 | 6 |
| β-Lapachone | 0.73 | 1.28 | 2 |
| Vitamin E | 2.37 | 25.8 | 11 |
| Arsenic Trioxide | 0.97 | 4.99 | 5 |
| Propyl Gallate | 3.24 | 10.6 | 3 |
| Quercetin | 2.22 | 6.07 | 3 |
| EGCG | 2.15 | 4.54 | 2 |

### Cytotoxicity of Vitamin C and FAN-NM-CH₃ Towards Different Cell Lines

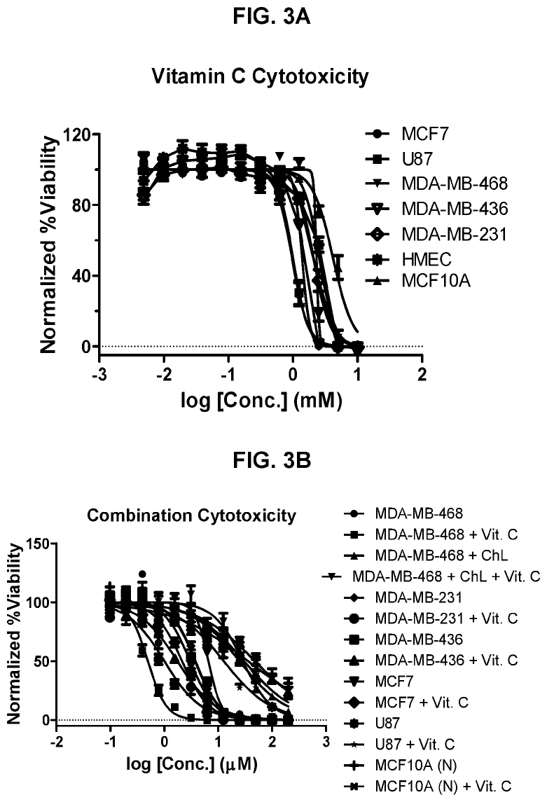

Pro-oxidants listed in Table 3 were initially screened in combination with FAN-NM-CH₃ in Triple-negative breast cancer MDA-MB-468 cells as well as normal epithelial cells (MCF 10A) for selectivity. Based on cytotoxicity data in FIG. 3A-B, Vitamin C in combination with FAN-NM-CH₃ showed the highest selectivity index (i.e., 73) for cancer cells in comparison to normal cells and Vitamin E has the second highest selectivity index (i.e., 11). These observations led to further screening of Vitamin C among other cancerous and non-cancerous cell lines.

TABLE 6
Cytotoxicity of Vitamin C in Different Cell Lines

| Cancer Type | IC₅₀ (mM)^{a} | |
|---|---|---|
| Breast Cancer | MCF7 | 0.97 ± 0.07 |
| Glioblastoma | U87 | 1.00 ± 0.1 |
| | MDA-MB-468 | 2.33 ± 0.3 |
| Triple-negative Breast Cancer | MDA-MB-436 | 1.39 ± 0.1 |
| | MDA-MB-231 | 2.23 ± 0.3 |
| Normal Epithelial | HMEC | 2.67 ± 0.4 |
| | MCF10A | 4.20 ± 0.4 |

^{a}The IC₅₀ of vitamin C in cancerous cells as well as normal cells when incubated for 48 h (n = 3, the values were determined by a nonlinear regression).

The data presented in Table 6 shows the IC₅₀ values of Vitamin C needed to kill 50% of the viable cells. Different types of cells exhibited different levels of sensitivity towards Vitamin C. To observe greater synergistic effect between the prodrug and the pro-oxidant without their individual toxicity, a maximum safe dose (MSD) was used in combination. The Maximum Safe Dose (MSD) refers to the highest dose of the pro-oxidant identified as safe, no discernible toxicity towards the tested cells. Combination of FAN-NM-CH₃ with the MSD doses of vitamin C in different cell lines produced synergistic anticancer effect (Table 7). The cell lines with higher MSD values had greater synergy as higher concentrations of Vitamin C potentially generate higher levels of H₂O₂ that accumulate in cells, which is sufficient to activate prodrug to produce toxic species leading to cancer cell death. We observed that cell lines reported to have higher endogenous H₂O₂ levels often lack catalase activity and can potentially accumulate even more H₂O₂ induced by Vitamin C. Normal epithelial MCF 10A cells with higher catalase activity, eliminated H₂O₂ concentrations. This led to improved selectivity of the combination strategy towards the cancer cells while sparing normal cells.

TABLE 7
Cytotoxicity of FAN-NM-CH₃ and Vitamin C at the Maximum
Safe Dose, Individually and Combined

| | | | FAN-NM-CH₃ | |
|---|---|---|---|---|
| | | | Combination | |
| | | Vitamin C | with | |
| | | Maximum | Vitamin C | |
| | FAN-NM-CH₃ | Safe Dose^{b} | Safe Dose | Fold |
| Cells | IC₅₀ (μM)^{a} | (mM) | IC₅₀ (μM)^{c} | Change^{d} |
| MDA-MBA-468 | 3.02 ± 0.2 | 1.00 | 0.49 ± 0.05 | 6 |
| MCF7 | 6.60 ± 0.5 | 0.25 | 2.14 ± 0.3 | 3 |
| U-87 MG | 24.83 ± 1.5 | 0.30 | 12.73 ± 0.8 | 2 |
| MDA-MB-231 | 3.45 ± 0.3 | 1 | 0.95 ± 0.1 | 4 |
| MDA-MB-436 | 3.65 ± 0.2 | 1 | 1.56 ± 0.1 | 2 |
| MDA-MB-468 + | 30.17 ± 2.3 | 1 | 28.66 ± 2.4 | 1 |
| Chlorambucil | | | | |
| MCF 10A (normal | 48.01 ± 3.5 | 1.25 | 35.87 ± 2.6 | 1 |
| cells) | | | | |

^{a}IC₅₀ of the prodrug alone when incubated for 48 h (n = 3, the IC₅₀ values were determined by a nonlinear regression).
^{b}The Maximum Safe Dose (MSD) refers to the highest dose of vitamin C identified as safe, exhibiting no discernible toxicity towards the tested cells.
^{c}IC₅₀ of the prodrug when combined with MSD of the vitamin C in cancerous cells as well as normal cells when incubated for 48 h (n = 3, the IC₅₀ values were determined by a nonlinear regression).
^{d}Fold Change: Fold change is the ratio of IC₅₀ value of the prodrug alone to that of the combination when incubated for 48 h (n = 3, the IC₅₀ values were determined by a nonlinear regression).

### Ascorbic Acid Demonstrates Potent Toxicity Towards Cancer Cells While Sparing Normal Cells

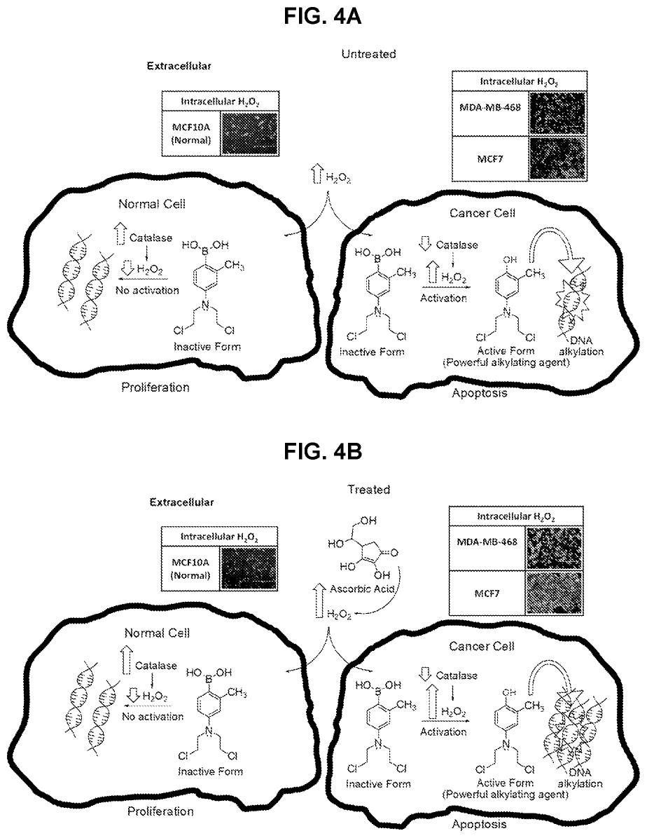

Its exceptional sensitivity to various cancer cell lines has garnered significant attention, leading researchers to delve deeply into its potential in synergistic therapeutic combinations with other cancer medications. Multiple studies have emphasized that elevated concentrations of ascorbic acid can distinctly induce apoptosis in cancer cells. Notably, certain cancer cell lines like MDA-MB-468, MCF7, and U-87 MG demonstrate higher levels of H₂O₂ compared to normal human breast epithelial cells (MCF 10A) due to reduced catalase activity in cancer cells. This discrepancy in H₂O₂ levels presents an opportunity for an additive effect when combining ascorbic acid with other cancer drugs. Ascorbic acid itself displays efficacy in eradicating MDA-MB-468, MCF7, and U-87 MG cells through H₂O₂-mediated cell death, contributing to its extensive exploration in combination therapies. However, the potential of ascorbic acid as an H₂O₂-generating agent to activate antineoplastic agents, such as of FAN-NM-CH₃, has not been thoroughly investigated. This approach holds promise for improving selectivity towards cancer cells and minimizing off-target effects. A schematic outline of the proposed mechanism is shown in FIG. 4A-B.

Before establishing the combination dose, it is imperative to investigate the cytotoxicity of ascorbic acid to define the uppermost safe dosage for each specific cell line. Extensive experimentation led to the determination of the half-maximal inhibitory concentration (IC₅₀) of ascorbic acid for MDA-MB-468, MCF7, U-87 MG, and MCF 10A cells, resulting in values of 2.3 mM, 1 mM, 1 mM, and 4.2 mM respectively (refer to FIG. 3A). Concentration-dependent analyses reveal that concentrations of 1 mM or less exhibit non-toxic effects on MDA-MB-468 tumor cells and MCF 10A normal cells, while doses of 0.25 mM and 0.3 mM are deemed safe for MCF7 and U-87 MG cells, respectively. To establish a synergistic interaction between FAN-NM-CH₃ and ascorbic acid, the strategy is to use the maximum safe doses of both compounds. The insights derived from FIG. 2A-C played a pivotal role in determining the maximum safe doses of FAN-NM-CH₃ (1 μM, 2 μM, 5 μM) and ascorbic acid (1 mM, 0.25 mM, 0.3 mM, and 1.25 mM), aiming to achieve a distinct combined effect and elucidate the contribution of ascorbic acid-induced activation of the prodrug FAN-NM-CH₃.

### The Combination of Ascorbic Acid Enhances the Potency of FAN-NM-CH₃

We utilized information from FIG. 2A to expose MDA-MB-468, MCF7, U-87 MG, and MCF 10A cells to safe doses of ascorbic acid: 1 mM, 0.25 mM, 0.3 mM, and 1.25 mM, respectively. Previous scientific studies consistently indicate that ascorbic acid treatment boosts H₂O₂ levels in cancer cells. This rise in H₂O₂ possesses the potential to deliberately trigger FAN-NM-CH₃'s activation in cancer cells, particularly due to their lesser catalase activity compared to regular cells. In our joint assay, cells were treated with ascorbic acid for an hour before introducing FAN-NM-CH₃. Following a 48-hour incubation period, the IC₅₀ values exhibited a considerable decrease in comparison to the use of FAN-NM-CH₃ alone. Following 48 hours of incubation, the IC₅₀ values showed a significant reduction compared to the IC₅₀ value of FAN-NM-CH₃ alone, with a 6-fold decrease for MDA-MB-468 cells, a 3-fold decrease for MCF7 cells, and a 2-fold decrease for U-87 MG cells. The new average IC₅₀ values for MDA-MB-468, MCF7, and U-87 MG cells were determined as 0.5 μM, 2.1 μM, and 12.7 μM, respectively. Notably, no notable toxicity was observed in normal MCF 10A cells, as the IC₅₀ value changed from 48 μM to 36 μM.

These findings suggest a cooperative effect between ascorbic acid and FAN-NM-CH₃, which necessitates deeper investigation. Previous research spotlighting ascorbic acid's function as an H₂O₂ generator in cancer cells lends credence to the concept that heightened H₂O₂ levels can selectively trigger arylboronates like the prodrug FAN-NM-CH₃ in cancer cells. This combined strategy has the potential to augment the safety of normal cells due to their elevated catalase activity, decreased reactive oxygen species (ROS) levels, and diminished prodrug dosage. These discoveries significantly enrich our understanding of the synergistic impacts arising from amalgamated dosages on both malignant and non-malignant cell populations.

The Synergistic Effect Between the Maximum Safe Dose of Ascorbic Acid and FAN-NM-CH₃, Coupled with Effective Quenching by Catalase

Previous data from FIG. 2A and Table 4 provided initial evidence supporting the presence of a combination effect between ascorbic acid and FAN-NM-CH₃. To validate this synergistic effect and attribute it to the increased levels of extracellular hydrogen peroxide induced by ascorbic acid treatment, catalase was introduced to the reaction with the intention of potentially catalyzing the decomposition of hydrogen peroxide and neutralizing the combination effect.

The selection of concentration ranges for both ascorbic acid and FAN-NM-CH₃ explored the boundaries and extent of the observed synergistic effect, despite the maximum safe dose being fixed for each cell line. Concentration ranges of 4 mM to 0.25 mM for ascorbic acid and 5 μM to 0.5 μM for the prodrug were chosen.

High doses of ascorbic acid (4 mM, 2 mM, and 1 mM) exhibited significant toxicity towards MCF7 and U-87 MG cells. A dose of 1 mM was found to be safe for MDA-MB-468 and MCF 10A cells, while 0.25 mM was safe for MCF7 and U-87 MG cells. The addition of catalase effectively quenched the toxicity induced by ascorbic acid in all samples. At a dose of 5 M, the prodrug FAN-NM-CH₃ displayed high toxicity towards MDA-MB-468 cells and some level of toxicity towards all tumor cells except for MCF 10A. The toxicity decreased as the concentration of FAN-NM-CH₃ was reduced, with doses of 1 μM and 0.5 μM showing no toxicity. The addition of catalase did not affect the toxicity of FAN-NM-CH₃ alone, possibly due to its limited ability to permeate the cells and quench intracellular H₂O₂. However, catalase effectively quenched the toxicity of ascorbic acid, which is known to release H₂O₂ extracellularly.

Combining high doses of ascorbic acid (4 mM, 2 mM, and 1 mM) with any dose of FAN-NM-CH₃ resulted in extreme toxicity towards all cells, as ascorbic acid itself exhibited greater cytotoxicity. The presence of catalase in these combination reactions effectively quenched the toxicity caused by ascorbic acid but not by FAN-NM-CH₃. To establish a clear synergistic effect and quantify the quenching by catalase, the maximum safe doses of ascorbic acid (1 mM, 0.25 mM, 0.3 mM, and 1.25 mM for MDA-MB-468, MCF7, U-87 MG, and MCF 10A, respectively) were combined with FAN-NM-CH₃ at doses of 1 μM, 2 μM, 5 μM, and 1 μM, respectively. As expected and consistent with the previous data, neither of the individual compounds exhibited toxicity alone. However, the combination of the two resulted in significant toxicity towards MDA-MB-468 and MCF7 cells, with average cell viability of 20% and 50%, respectively. U-87 MG cells experienced less toxicity, with an average cell viability of 65%. MCF 10A cells did not show noticeable cytotoxicity in the combination. The toxic effect was effectively quenched by catalase in all combinations. These experiments clearly demonstrated that the combination effect is mediated by ascorbic acid through increased levels of H₂O₂. The inclusion of catalase and its quenching action confirmed that the elevated levels of reactive oxygen species (ROS) were indeed H₂O₂.

Further experiments were required to investigate the catalase activity of different cancerous and non-cancerous cells to gain a deeper understanding and establish the pro-oxidative nature of ascorbic acid. This knowledge is crucial in considering ascorbic acid as a potential candidate for combination therapies with other ROS-activated prodrugs, such as FAN-NM-CH₃, and to assess innate design in both tumor and normal cells, where tumor cells lack the ability to metabolize H₂O₂ while normal cells possess this capability.

TABLE 8
Cytotoxicity of FAN-NM-CH³ in Different Cell Lines, Maximum
Safe Dose of Ascorbic Acid, and Cytotoxicity in Combination

| | | Vitamin C | FAN-NM-CH₃ | |
|---|---|---|---|---|
| | | Maximum | Combination | |
| | | Safe | with Vitamin | |
| | FAN-NM-CH₃ | Dose^{b} | C Safe Dose | Fold |
| Cells | IC₅₀ (μM)^{a} | (mM) | IC₅₀ (μM)^{c} | Change^{d} |
| MDA-MBA-468 | 3.02 ± 0.2 | 1.00 | 0.49 ± 0.05 | 6 |
| MCF7 | 6.60 ± 0.5 | 0.25 | 2.14 ± 0.3 | 3 |
| U-87 MG | 24.83 ± 1.5 | 0.30 | 12.73 ± 0.8 | 2 |
| MCF 10A | 48.01 ± 3.5 | 1.25 | 35.87 ± 2.6 | 1 |

Variation in Catalase Activity and its Effects on Extracellular H₂O₂ Removal

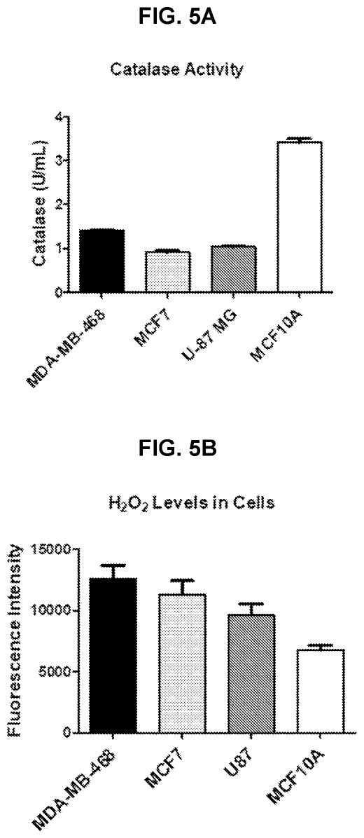

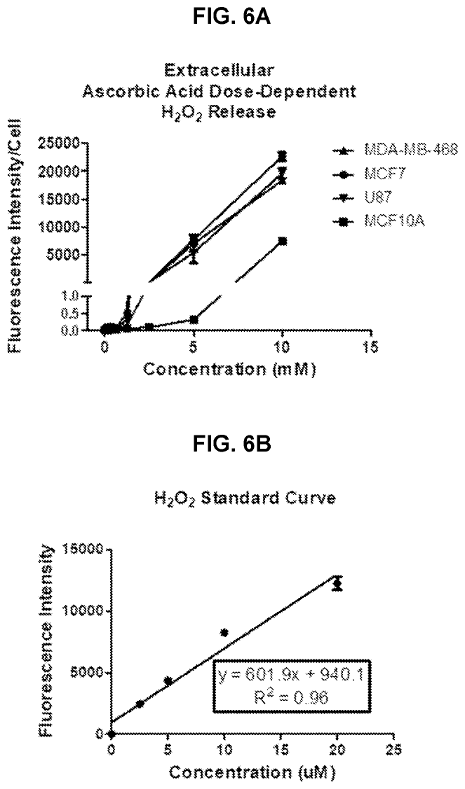

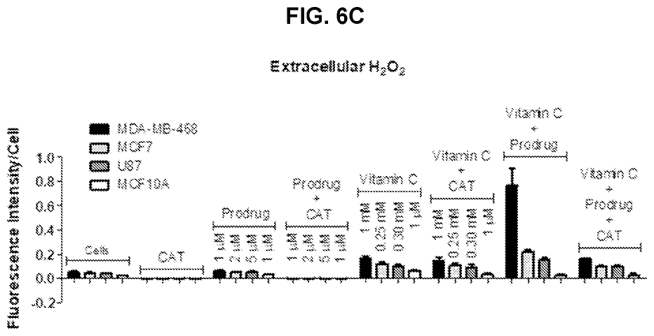

The diverse reactions of distinct cell lines to ascorbic acid prompt an investigation into whether this variability stems from differing H₂O₂ levels generated by the acid. If this assumption holds, a closer examination of the enzyme accountable for catalyzing the decomposition of H₂O₂ becomes necessary. Several kinetic models propose the vital role of catalase, a key antioxidant enzyme, in mitigating H₂O₂ within cells. Thus, the current study aims to assess catalase activity across various cancerous and non-cancerous cell types. The findings displayed in FIG. 5A-B reveal that normal cells exhibit an average catalase activity of 3.4 U/mL (1 mL of cell lysate; 1×10⁶ cells). A noticeable threefold contrast in catalase activity arises between cancerous and non-cancerous cells. While no significant deviation in catalase activity was evident among the three examined cancer cell lines, existing research suggests a broader range of H₂O₂ removal capabilities among diverse cancer cell lines, intricately linked with their catalase activity. Despite the relatively subtle differences among these three cancer cell lines, MCF7 cells notably exhibit the lowest catalase activity at 0.9 U/mL, followed by U-87 MG cells with an average activity of 1 U/mL, and finally MDA-MB-468 cells at 1.4 U/mL. This diversity in catalase activity may exert a more profound influence when considering a wider spectrum of cancer cell lines, potentially impacting their ability to break down H₂O₂. Interestingly, exposure to 5 μM H₂O₂ (FIG. 6A-C) uncovers a strong connection between the ability to clear extracellular H₂O₂, responsiveness to ascorbic acid, and catalase activity. In summary, these findings underscore that cell with heightened catalase activity, like MCF 10 A cells, display an enhanced capability to degrade H₂O₂, resulting in decreased sensitivity to ascorbic acid and a lesser buildup of H₂O₂. Conversely, cells featuring lower catalase activity, such as MCF-7, U-87 MG, and MDA-MB-468, manifest reduced potential to metabolize H₂O₂, heightened susceptibility to ascorbic acid, and augmented H₂O₂ accumulation.

Ascorbic Acid Induced High Levels of Extracellular H₂O₂ in Cancer Cells

Having established a direct correlation between lower catalase activity and reduced cellular ability to metabolize hydrogen peroxide (H₂O₂), further investigation was required to validate the dependence of H₂O₂ release on ascorbic acid concentration in different cancer cells. Simultaneously, another experiment was conducted to quantify alterations in H₂O₂ levels under diverse circumstances. To validate the identity of the reactive oxygen species (ROS) as H₂O₂, catalase was introduced to quench the generated ROS. The outcome presented in FIG. 2B demonstrates that catalase effectively counteracted the cooperative impact of ascorbic acid and FAN-NM-CH₃, indicating shifts in H₂O₂ levels that hindered the synergistic blend. To measure extracellular H₂O₂ in different cells, the Amplex Red assay was utilized, employing Amplex Red reagent that reacts with H₂O₂ to produce the red-fluorescent byproduct resorufin, characterized by maximum excitation/emission wavelengths of 560/590 nm. FIG. 6A demonstrates that increasing the concentration of ascorbic acid at millimolar (mM) scale led to a significant increase in H₂O₂ levels in the extracellular regions of cancer cells, while normal cells exhibited minimal H₂O₂ generation even at the highest dosage. The most substantial change in H₂O₂ levels was observed in MCF7 cells, with over 20 μM of H₂O₂ generated. MDA-MB-468 cells exhibited similar but lower levels compared to MCF7, while U-87 MG cells showed the least H₂O₂ levels. Interestingly, the escalation in H₂O₂ levels with higher ascorbic acid concentrations was not consistently proportional to the cell count, and higher concentrations prompted cell death, as indicated in FIG. 1A. A similar effect was observed for MDA-MB-468 cells, where 2 mM resulted in 50% cell death. Normalizing the data based on the percentage of viable cells revealed that the signals would increase and become closer to double at the IC₅₀ concentrations. These signals would be even higher after normalization for higher concentrations. This prompts the question of why a lower number of cells would produce a higher signal. It has been reported that dying cells generate a burst of ROS that propagates as a survival signal to surrounding cells. This finding supports the sudden increase in H₂O₂ levels at higher concentrations, which are toxic to cells. In normal cells, changes in H₂O₂ levels were limited when the concentration of ascorbic acid was altered. MCF 10A cells exhibited minimal change in H₂O₂ levels at lower concentrations of ascorbic acid, with slight changes observed beyond the IC₅₀ concentration of 4 mM.

FIG. 6C demonstrates that all cancer cells had some level of extracellular H₂O₂ (~2 μM), with normal cells exhibiting the lowest levels. Strikingly, when cells were exposed to 5 μM H₂O₂ externally (FIG. 6C), variations in H₂O₂ metabolism among different cells were observed, aligning with the earlier experiment (FIG. 5A-B). The findings were consistent with the roles of catalase activity; MCF 10A cells efficiently metabolized H₂O₂, leading to lower detection levels. Among cancer cell lines, the ability to process the same H₂O₂ level was reduced. Upon closer comparison with FIG. 3, MCF7 cells, exhibiting the lowest catalase activity, displayed the highest extracellular H₂O₂ levels, followed by U-87 MG cells and then MDA-MB-468 cells. These H₂O₂ levels inversely correlated with catalase activity. However, observed variations in extracellular H₂O₂ levels at their respective maximum safe ascorbic acid doses in FIGS. 6A and 6C, around 10 μM, 7 μM, 7 μM, and 5 μM H₂O₂ for MDA-MB-468, MCF7, U-87 MG, and MCF 10A cells, respectively highlight the challenge of directly linking catalase activity and H₂O₂ metabolism driven by ascorbic acid dosage. Additionally, catalase effectively quenched extracellular H₂O₂ at the fixed maximum safe ascorbic acid doses for all cell lines, including MCF 10A. However, as higher levels of H₂O₂ were generated at these concentrations, catalase's efficacy diminished. Moreover, the presence of high H₂O₂ levels from dying cells, as reported, hindered observable quenching at these concentrations. Consequently, the combination was examined at lower ascorbic acid concentrations, where H₂O₂ levels were lower. As expected, a smaller number of cells were affected by the synergistic effect, allowing catalase to quench some H₂O₂. Interestingly, the presence of ascorbic acid hindered catalase's effectiveness, leading to a more pronounced quenching effect at lower concentrations compared to higher ones. Furthermore, the data implies that higher H₂O₂ levels enhanced the viability and efficiency of this combined approach. Upon catalase addition, the signal decreased for all cell lines except for MCF 10A. In summary, these findings, in line with previous experiments, supported employing the maximum safe ascorbic acid dose alongside FAN-NM-CH₃ to achieve an effective synergistic outcome comparable to a ten-fold prodrug dosage alone. Additionally, the accessibility and efficacy of this combination were likely bolstered by the increased abundance of H₂O₂. The observed changes in extracellular H₂O₂ levels likely correlated with intracellular levels. Thus, it was essential to determine if extracellular H₂O₂ generation also led to intracellular H₂O₂ increase and to examine potential differences from extracellular H₂O₂ deposition.

Exploring the Impact of Ascorbic Acid on Intracellular H₂O₂ Levels

Hydrogen peroxide (H₂O₂) is a small molecule without charge, enabling it to traverse cellular membranes and establish equilibrium between extracellular and intracellular concentrations. Given H₂O₂'s properties and the cellular context, studying extracellular H₂O₂ concentrations offers insights into intracellular levels, as exemplified in our prior experiment (FIG. 4). FIG. 7A data indicates that increased ascorbic acid concentrations correlate with elevated intracellular H₂O₂ levels. However, this effect was notably prominent in cancer cells while sparing normal cells. The rise peaked and declined at the highest concentrations, coinciding with cell death. These findings imply a detectable exchange of H₂O₂ between extracellular and intracellular spaces, surpassing what was previously reported. The cellular antioxidative defense system rapidly consumes externally added H₂O₂, leading to a gradient approximately 7-10 times lower. Surprisingly, our study suggests a mere 2-fold reduction in intracellular H₂O₂ concentration in MDA-MB-468 cells. Moreover, the correlation wasn't observed in MCF7 and U-87 MG cells at the concentration in FIG. 7C, potentially explaining the combination's limited effectiveness. When 2.5 μM of external H₂O₂ was introduced, a similar intracellular metabolism pattern to extracellular samples emerged. Higher catalase activity corresponded with lower intracellular H₂O₂ levels. Compared to MCF7 and U-87 MG cells, MDA-MB-468 cells exhibited lower H₂O₂ levels. Among the tested cell types, MCF7 cells displayed the highest endogenous H₂O₂ levels, followed by U-87 MG cells, then MDA-MB-468 cells. Normal cells exhibited the lowest signal consistently maintained throughout the experiment. Addition of extracellular catalase notably decreased intracellular H₂O₂ accumulation across all samples, indicating a gradient allowing H₂O₂ exchange between cellular environments. FIG. 7C reveals slight H₂O₂ level variations between MCF7 and U-87 MG cells under 0.25 mM and 0.3 mM ascorbic acid treatment, respectively. However, MDA-MB-468 cells exhibited a significant threefold increase compared to untreated samples. Intriguingly, intracellular H₂O₂ results didn't fully correspond with extracellular observations, possibly due to variations in H₂O₂ equilibrium and permeation among cell types or interactions with cellular components. Probe sensitivity may also play a role, as intracellular H₂O₂ levels might not yield detectable signals with this assay.

### DNA Damage Detected by Alkaline Comet Assay

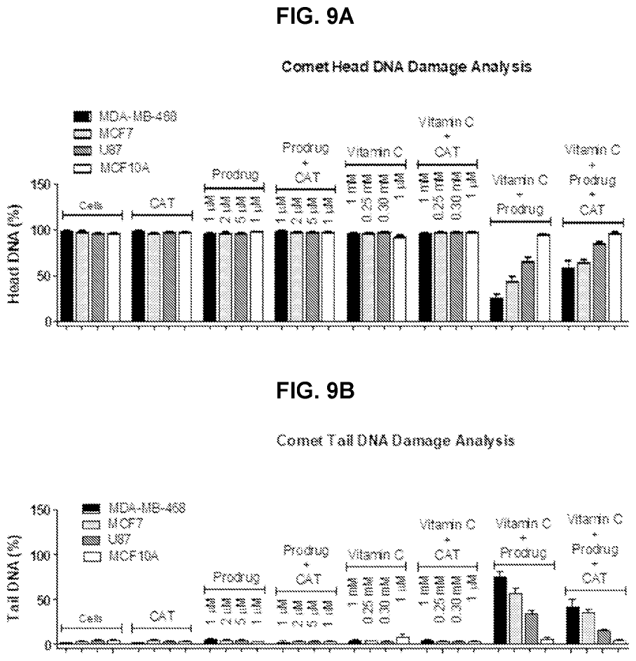

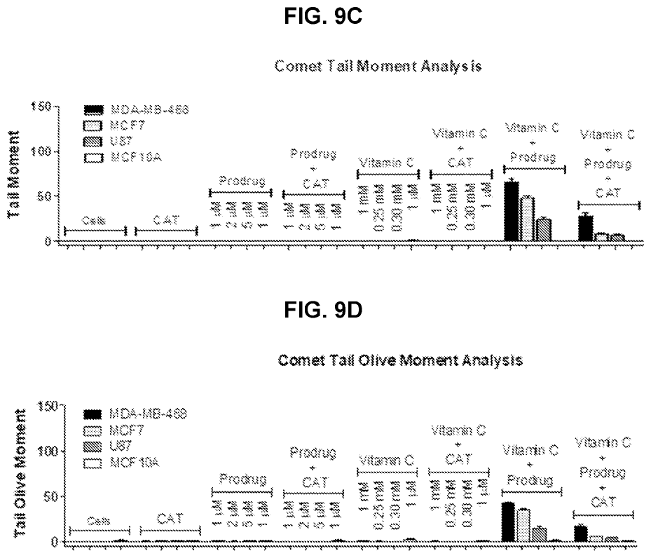

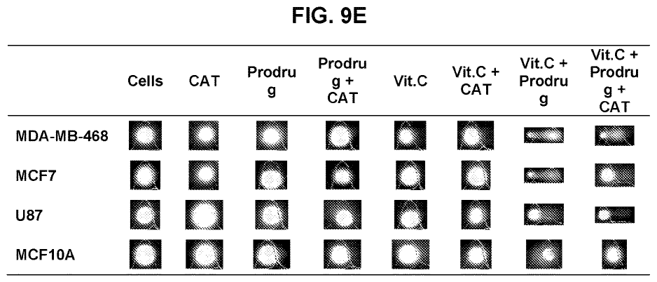

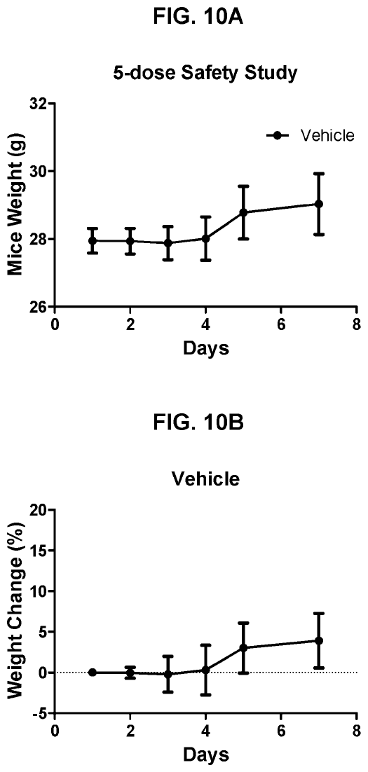

DNA damage analysis was conducted on a group of 30 comets using TriTek CometScore Software. Four major parameters, namely Head DNA (%), Tail DNA (%), Tail Moment, and Olive Tail Moment, were examined in FIG. 9A-E to assess the extent of damage. Higher values for Tail DNA, Tail Moment, and Olive Tail Moment indicate greater DNA damage, while Head DNA signifies the opposite. Tail Moment and Olive Tail Moment were utilized as accurate indicators of DNA damage. Tail Moment is calculated by multiplying the Tail length by the fraction of Tail DNA (%), while Olive Tail Moment is calculated by multiplying the Tail DNA (%) by the fraction of the difference between the mean values of Head and Tail DNA. The data presented in FIG. 9A-E demonstrates that Head DNA (%), Tail DNA (%), Tail Moment, and Tail Olive Moment remained relatively stable across various conditions, except for samples treated with a combination of ascorbic acid and FAN-NM-CH₃, as well as samples treated with catalase to counteract the combination effect. Individual maximum safe doses of ascorbic acid and FAN-NM-CH₃ did not show any observable DNA damage. In FIG. 9A, MDA-MB-468 cells exhibited a significant reduction in the percentage of Head DNA (indicating more DNA damage) to 24%. This percentage increased in samples with reduced synergistic effect, with MCF7 at 43%, U-87 MG at 66%, and the highest Head DNA observed in normal cells (MCF 10A) at 95%. These numbers increased when catalase was added, quenching the synergistic effect between ascorbic acid and FAN-NM-CH₃. FIG. 9B illustrates that Tail DNA (%) was higher in samples displaying greater synergistic effect, with MDA-MB-468 having the highest at 75%, MCF7 at 57%, U-87 MG at 34%, and MCF 10A exhibiting the least damage at 6%. These numbers significantly decreased in samples with catalase treatment, which prevented the synergistic effect and reduced DNA damage. In FIG. 9C, Tail Moment was highest in samples with greater synergistic effect, with MDA-MB-468 cells at 65%, MCF7 at 48%, U-87 MG at 24%, and MCF 10A at 0.6%. Tail moments were substantially reduced when catalase was added to the system, quenching the synergistic effect. FIG. 9D presents the Tail Olive Moment (%), with MDA-MB-468 cells experiencing the highest synergistic effect and displaying the highest Tail Olive Moment at 42%, followed by MCF7 cells at 35%, U-87 MG cells at 14%, and MCF 10A cells at only 1.5%. These values decreased when catalase was introduced to the system, effectively suppressing the synergic effect, and preventing DNA damage. These results align closely with previous findings and strongly support the coherence of the strategy. Ascorbic acid and the prodrug, when administered at their maximum safe doses, have demonstrated safety. They produce a potent anticancer effect that selectively targets cancer cells while sparing normal cells.

Recent advancements in cancer research have led to the discovery of numerous potential anticancer agents worldwide. These agents are being extensively studied both in combination therapies and as monotherapeutic agents. However, the complex nature of cancers has posed significant challenges in their treatment. Tumor cells, including MDA-MB-468, MCF7, and U-87 MG, exhibit characteristics such as poor antigrowth signals, high metastatic risk, uncontrolled proliferation, angiogenesis, and evasion of apoptosis through various cellular pathways. To address these challenges, studies have demonstrated that combination therapies offer a more sustained anticancer effect compared to monotherapies. Animal studies have consistently shown that drug combinations provide greater benefits than single-agent therapies. Despite the accumulating evidence supporting the potential of combination therapies, many pharmaceutical companies tend to focus on developing single agents, which often results in failure to obtain FDA approval. The identification of potent drug combinations poses scientific and logistical challenges that researchers must overcome through innovative and strategic approaches. One such approach involves the use of ROS-responsive prodrugs to enhance the effectiveness of combination therapies. Several research groups are actively investigating these ROS-responsive anticancer agents. For example, a series of aryl boronated nitrogen mustard-containing prodrugs have been developed that selectively react with H₂O₂ in cancer cells. While other researchers have explored the combination of ascorbic acid with other anticancer drugs, ascorbic acid has consistently been used at high doses throughout the research. Its role as an anticancer agent has been heavily relied upon, necessitating high doses to generate significant levels of H₂O₂ that can cause DNA damage and cell death. However, some researchers have expressed concerns about the widespread use of high doses of ascorbic acid by complementary and alternative medicine practitioners. These concerns arise from the lack of reporting and attribution of adverse effects and side effects directly to ascorbic acid.

To address these concerns, this study proposes a safer approach by combining ascorbic acid with other ROS-responsive prodrugs. Specifically, a highly potent prodrug called FAN-NM-CH₃, which contains aryl boronated nitrogen mustard has shown remarkable effectiveness, being 10 times more potent (IC₅₀=3.1 μM) against the MDA-MB-468 cell line compared to chlorambucil (IC₅₀=34.4 μM). Furthermore, it has demonstrated superior in vivo efficacy, selectivity, reduced adverse effects, and improved pharmacokinetics. When ascorbic acid is combined with the maximum safe dose of FAN-NM-CH₃, it enhances drug toxicity, leading to approximately 80% cell death in MDA-MB-468 cells, 50% in MCF7 cells, and 60% in U-87 MG cells, while sparing MCF 10A cells. This synergistic effect is equivalent to the toxicity achieved with a 10 μM, 7 μM, and 20 μM dose of FAN-NM-CH₃ alone in MDA-MB-468, MCF 7, and U-87 MG cancer cells, respectively, over 48 hours. In terms of fold differences, the combination of maximum safe doses was 10-fold, 3-fold, and 4-fold lower than the doses of FAN-NM-CH₃ alone in MDA-MB-468, MCF 7, and U-87 MG cancer cells, respectively, to produce the same level of toxicity towards cancer cells. The combination approach significantly reduces the drug dosage while maintaining selectivity and effectiveness. By introducing ascorbic acid, not only is the IC₅₀ of the prodrug reduced, but the efficacy of the drug is also improved by selectively delivering H₂O₂ to cancer cells and activating the prodrug within those cells.

Furthermore, this combination therapy can potentially be enhanced by using higher doses of ascorbic acid and FAN-NM-CH₃, considering that both compounds exhibit anticancer properties that can kill cancer cells while sparing normal cells. Given that both agents are used at non-lethal doses, it is less likely that adverse effects will be observed based on the presented results. According to Humphrey et al., if there is a pharmacodynamic interaction between two agents, meaning that one enhances the toxicity of the other, such drug combinations can proceed directly to phase II studies if animal studies have demonstrated their safety in combination. However, this aspect is yet to be determined for FAN-NM-CH₃ and ascorbic acid. These findings suggest that the prodrug FAN-NM-CH₃ holds promise as a diverse anticancer agent when combined with ascorbic acid, offering improved selectivity, efficacy, and reduced dosage. These experiments can serve as a foundation for further research in preclinical models, facilitating the development of enhanced treatment strategies.

### In Vivo Safety Study

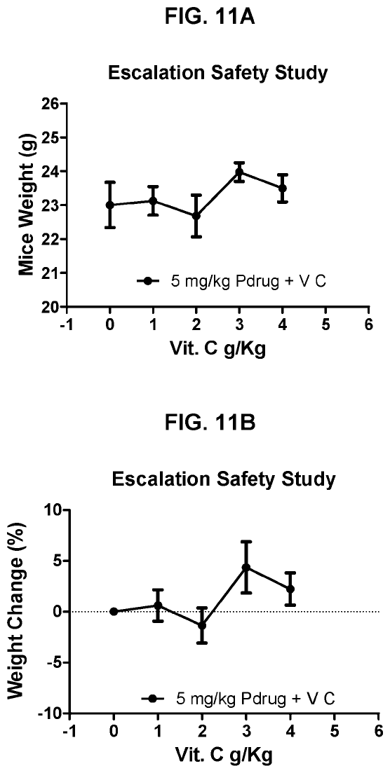

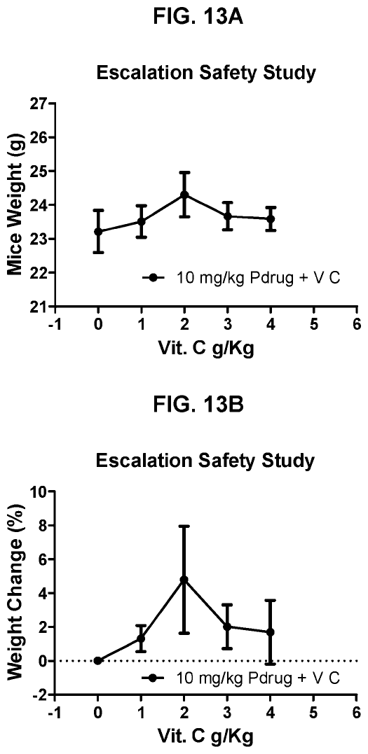

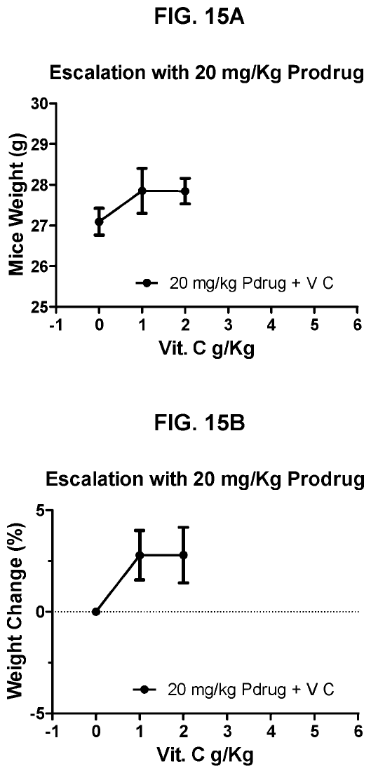

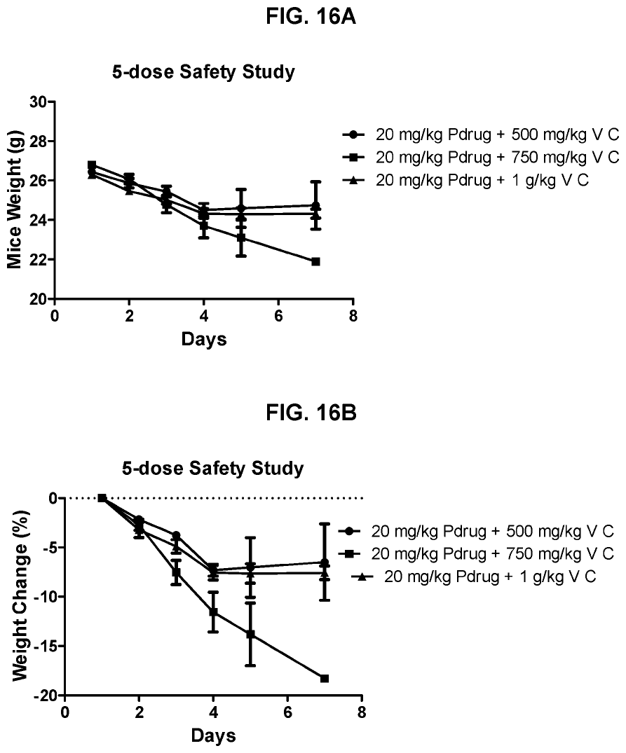

The toxicity of the combination was evaluated in vivo in comparison to the Vehicle, Vitamin C, and the prodrug alone. The escalation safety study was carried out with fixed 5 mg/kg, 10 mg/kg, and 20 mg/kg (FIGS. 11A-B, FIGS. 13A-B, and FIGS. 15A-B) of FAN-NM-CH₃ while escalating 1 g/kg of the Vitamin C dosage until serious adverse events were observed or the maximum Vitamin C dosage of 4 g/kg was reached. The results are summarized in Table 9. No obvious signs of toxicity were observed with Vitamin C dosage up to 4 g/kg when it was in combination with 5 mg/kg and 10 mg/kg dosage of the prodrug. However, combination of Vitamin C with 20 mg/kg prodrug showed symptoms of toxicity including weight loss, reduced activity, and death.

TABLE 9
Mouse Survival Rate After Escalation Treatment

| | Vitamin C (g/kg) | | | |
|---|---|---|---|---|
| Prodrug (mg/kg) | 1 | 2 | 3 | 4 |
| 5 | 3/3 | 3/3 | 3/3 | 3/3 |
| 10 | 3/3 | 3/3 | 3/3 | 3/3 |
| 20 | 3/3^{a} | 3/3^{a} | ⅓^{a} | n.d |

^{a}Symptoms of toxicity were observed, including weight loss, loss of appetite, and reduced activity levels.
Three mice per group were administered with the combination of the prodrug and Vitamin C.

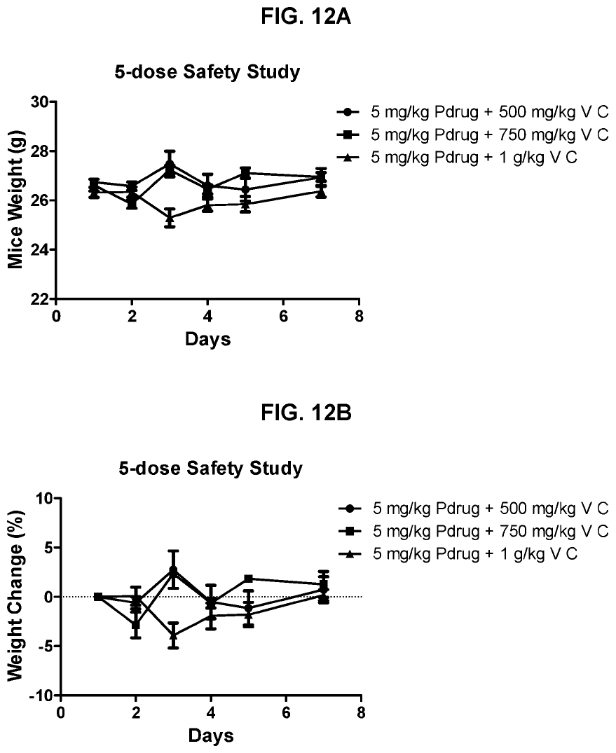

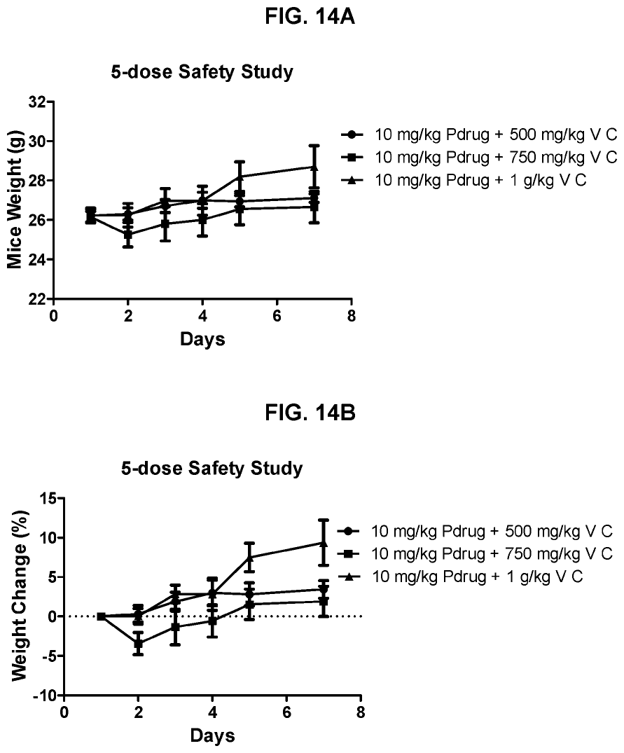

To further understand the delayed-onset toxicity prior to in vivo efficacy study, a repeated-dose toxicity study was conducted. Mice were treated daily via IP for one week, and the body weights were measured daily. 9 groups (3 mice/group) of mice were used for this study. Each group of mice were treated daily either with vitamin C alone (500 mg/kg, 750 mg/kg, or 1000 mg/kg), prodrug along (5 mg/kg, 10 mg/kg, or 20 mg/kg), or combination of vitamin C and prodrug. For the combination therapy, each dosage of prodrug (5 mg/kg, 10 mg/kg, or 20 mg/kg) was administered in combination with three dosages of Vitamin C (500 mg/kg, 750 mg/kg, and 1000 mg/kg). These dosages were fixed and administered via IP for 5 days (FIG. 12A-B, FIGS. 14A-B, and 16A-B). Results are summarized in Table 10. No toxicity was observed with combination of Vitamin C (500 mg/kg, 750 mg/kg, and 1000 mg/kg) with 5 mg/kg or 10 mg/kg prodrug group. An increase in body weight was observed for all the mice treated with combination of 5 mg/kg or 10 mg/kg prodrug with vitamin C ranging from 500-1000 mg/kg doses. However, significant weight loss was observed for combination of Vitamin C (500 mg/kg, 750 mg/kg, or 1000 mg/kg) with 20 mg/kg prodrug dosage. Thus, to observe a synergistic effect between the prodrug and Vitamin C in combination rather than their individual effect in a monotherapy, a lower dose of 3 mg/kg of the prodrug with 500 mg/kg dose of the Vitamin C was used for the in vivo efficacy study.

TABLE 10
Mouse Survival Rate After 5-dose Treatment

| | Vitamin C (mg/kg) | | |
|---|---|---|---|
| Prodrug (mg/kg) | 500 | 750 | 1000 |
| 5 | 3/3 | 3/3 | 3/3 |
| 10 | 3/3 | 3/3 | 3/3 |
| 20 | 3/3^{a} | 3/3^{a} | 3/3^{a} |

^{a}Symptoms of toxicity were observed, including weight loss, loss of appetite, and reduced activity levels.
Three mice per group were administered with the combination of the prodrug and Vitamin C.

### Vitamin C and FAN-NM-CH₃ Combination Leads to Tumor Reduction

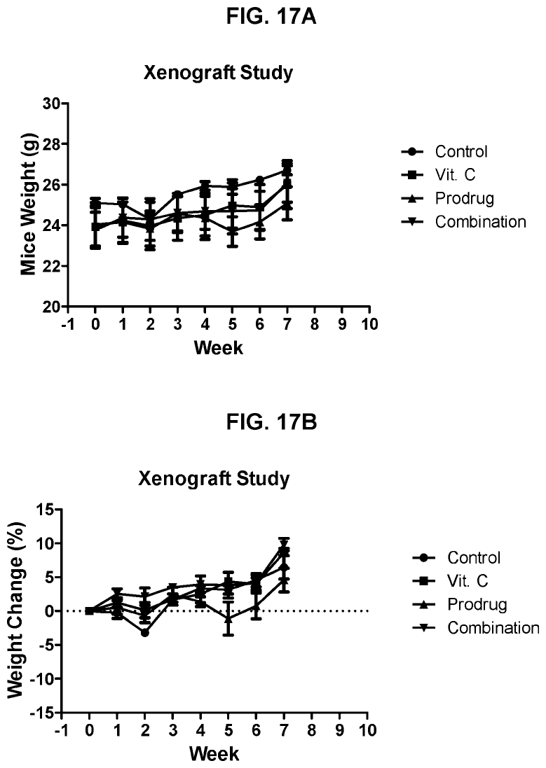

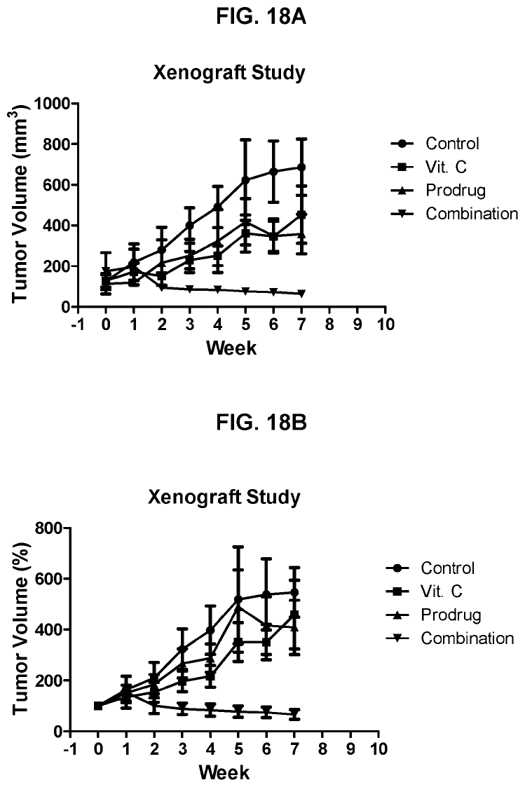

To investigate the in vivo efficacy of the combination therapy (the prodrug FAN-NM-CH₃ and vitamin C), the athymic nude mice were inoculated with the human MDA-MB-468 breast cancer cells subcutaneously. Tumors were developed in all mice within one week. The mice were divided into four groups (5 mice each), which were treated with vehicle, 500 mg/kg Vitamin C, 3 mg/kg FAN-NM-CH₃ (Prodrug), and the combination (Vitamin C+FAN-NM-CH₃ (Prodrug)). The weight of mice and size of tumors were measured weekly by caliper. The data are presented in FIGS. 17A-B and FIGS. 18A-B, respectively. After three weeks of treatments, both Vitamin C and the FAN-NM-CH₃ alone had observable inhibitory effects on the tumor size in comparison to the vehicle tumor size (FIGS. 18A-B). Combination of Vitamin C+Prodrug not only inhibited the tumor growth but reduced the tumor size after 3 weeks of treatment. In contrast, vehicle-treated mice showed significant tumor growth, reaching 250% of the initial tumor size in 3 weeks. All groups were monitored for symptoms of toxicity including changes in body weight, loss of appetite, reduced activity levels, treatment-related mortality, etc. Our data suggested that neither of the groups showed any signs of toxicity. Additionally, in vivo results demonstrated that a treatment of Vitamin C combined with the prodrug potently suppressed the tumor growth without any signs of toxicity.

What is claimed:

1. A combination therapeutic comprising:
a therapeutically effective amount of one or more antineoplastic agents, or pharmaceutically acceptable salts or esters thereof, and
a therapeutically effective amount of one or more pro-oxidants, or pharmaceutically acceptable salts or esters thereof.

2. The combination therapeutic of claim 1, wherein the one or more antineoplastic agents is an alkylating agent or a pro-drug of an alkylating agent.

3. The combination therapeutic of claim 1, wherein the one or more antineoplastic agents has a structure: 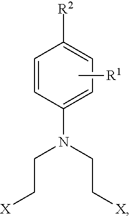
wherein
X, at each occurrence, is independently halo;
R¹, at each occurrence, is independently hydrogen, C₁₋₆alkyl, C₁₋₆alkylene, C₁₋₆haloalkyl, cyano, —OR^{1a}, —SR^{1a}, —CO₂R^{1a}, —C(O)R^{1a}, —SO₂R^{1b}, —N(R^{1b})₂, —CO₂N(R^{1b})₂, or —NO₂;
R² is BH(OR^{1b}), B(OR^{1b})₂, 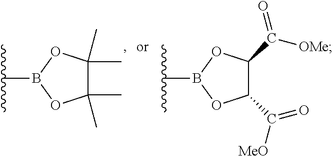
R^{1a}, at each occurrence, is independently hydrogen, C₁₋₆alkyl, or C₁₋₂haloalkyl; and
R^{1b}, at each occurrence, is independently hydrogen or C₁₋₆alkyl.

4. The combination therapeutic of claim 1, wherein the one or more antineoplastic agents is FAN-NM-CH₃.

5. The combination therapeutic of claim 1, wherein the one or more antineoplastic agents is a DNA crosslinking agent or a pro-drug of a DNA crosslinking agent.

6. The combination therapeutic of claim 1, wherein the one or more antineoplastic agent is selected from the group comprising: 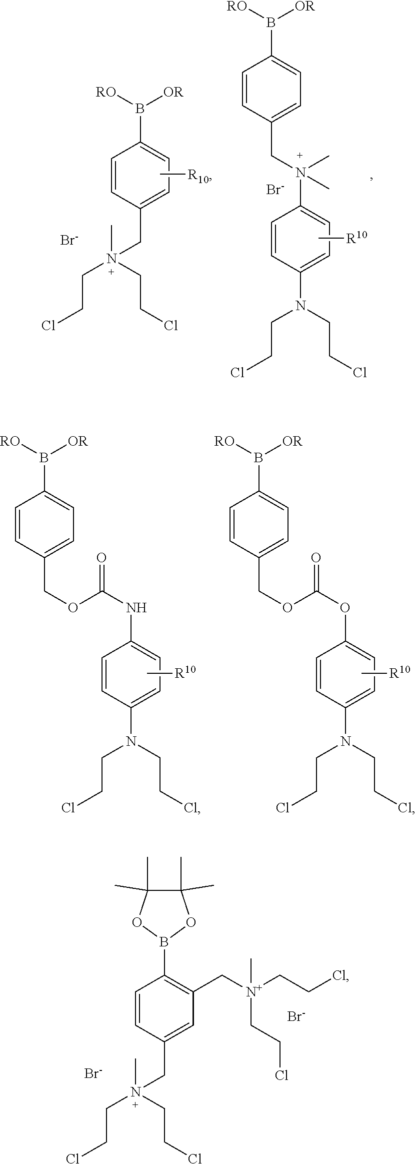 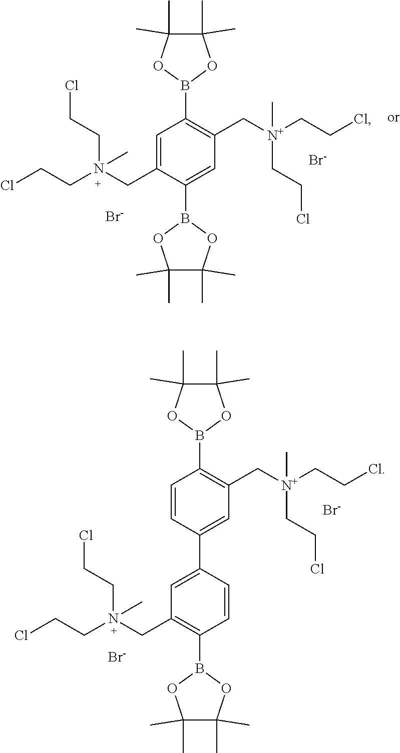
wherein R¹⁰, at each occurrence, is independently hydrogen, C₁₋₆alkyl, C₁₋₆alkylene, C₁₋₆haloalkyl, cyano, —OR^{1a}, —SR^{1a}, —CO₂R^{1a}, —C(O)R^{1a}, —SO₂R^{1b}, —N(R^{1b})₂, —CO₂N(R^{1b})₂, —NO₂, or —N(RR^{1b})—OR^{1a};
R^{1a}, at each occurrence, is independently hydrogen, C₁₋₆alkyl, or C₁₋₂haloalkyl; and
R^{1b}, at each occurrence, is independently hydrogen or C₁₋₆alkyl.

7. The combination therapeutic of claim 1, wherein the one or more antineoplastic agents comprise one or more chemotherapeutic agents.

8. The combination therapeutic of claim 7, wherein the one or more chemotherapeutic agents comprise adriamycin, anthracyclines, bleomycin, or cisplatin, their H₂O₂-activated prodrugs, or combinations thereof.

9. The combination therapeutic of claim 1, wherein the one or more pro-oxidants is a reactive oxygen species amplifying agent.

10. The combination therapeutic of claim 1, wherein the one or more pro-oxidants comprise compounds containing quinone moieties.

11. The combination therapeutic of claim 1, wherein the one or more pro-oxidants comprise vitamin C, polyphenol, hydrogen peroxide, carotenoids (Lutein, β-carotene, Astaxanthin, Fucoxanthin, β-Cryptoxanthin, Bixin, and lycopene), diallyl trisulfide (DATS), indomethacin (indo), Piperlongumine, Vitamine E, β-Lapachone, Plumbagin, Arsenic Trioxide, Quercetin, Cinnamaldehyde, Bisdemethoxycurcumin (Curcumin), (−)-Epigallocatechin gallate, Gensenosides (Rg3, Rh2), 2-methoxyestradiol, Emodin, β-phenylethyl isothiocyanate, nonsteroidal anti-inflammatory drugs (NSAIDs), or combinations thereof.

12. The combination therapeutic of claim 1, wherein the therapeutically effective amount of the one or more antineoplastic agents is 0.001-200 mg/kg.

13. The combination therapeutic of claim 1, wherein the therapeutically effective amount of the one or more pro-oxidants is 1-20000 mg/kg.

14. A method for treating a disease or disorder, the method comprising:
sequentially or simultaneously administering to a subject in need thereof
a therapeutically effective amount of one or more antineoplastic agents, or pharmaceutically acceptable salts or esters thereof, and
a therapeutically effective amount of one or more pro-oxidants, or pharmaceutically acceptable salts or esters thereof; and
repeating the administration until the disease or disorder is treated, ameliorated, or symptoms are reduced.

15. The method of claim 14, wherein the disease or disorder is a cancer.

16. The method of claim 14, wherein the disease or disorder is a solid cancer.

17. The method of claim 15, wherein the cancer is a breast cancer, a glioblastoma, leukemia, lung cancer, or renal cancer.

18. The method of claim 14, wherein the disease or disorder is a cancer associated with oxidative stress.

19. The method of claim 14, wherein the disease or disorder is an inflammatory disease.

20. The method of claim 19, wherein the inflammatory disease is arthritis.

21. The method of claim 14, wherein administering comprises intraperitoneal injection, intramuscular injection, subcutaneous injection, intravenous injection, intrathecal infusion, oral administration, or a combination thereof.

22. The method of claim 14, wherein the one or more pro-oxidants increase an amount of reactive oxygen species in a cancerous cell.

23. The method of claim 14, wherein the one or more antineoplastic agents is active in the presence of reactive oxygen species.

24. The method of claim 14, wherein a therapeutically effective amount of a one or more pro-oxidants and one or more antineoplastic agents reduces a malignant neoplasm size, volume, mass, or a combination thereof.

25. The method of claim 14, wherein the one or more pro-oxidants is added at a period of time prior to addition of the antineoplastic agent.

26. The method of claim 14, wherein the period of time is 1-3 hours.

27. Use of one or more antineoplastic agents and one or more pro-oxidants, or pharmaceutically acceptable salts or esters thereof, as a medicament for the treatment of cancer or an inflammatory disease in subject in need thereof.

28. A kit comprising:
one or more antineoplastic agents, or pharmaceutically acceptable salts or esters thereof;
one or more pro-oxidants, or pharmaceutically acceptable salts or esters thereof;
optionally a device or means for administering the antineoplastic agent and pro-oxidant;
optionally tamper resistant packaging; and
optionally, a label or instructions for use thereof.
